(19) United States
(12) Patent Application Publication
(10) Pub. No.: US 2021/0388070 A1
(43) Pub. Date: Dec. 16, 2021

(54) CO-ADMINISTRATION OF A HYALURONIDASE AND ANTI-C5 ANTIBODY FOR TREATMENT OF COMPLEMENT-ASSOCIATED CONDITIONS

(71) Applicant: Alexion Pharmaceuticals, Inc., Boston, MA (US)

(72) Inventors: Andrew Denker, Ardmore, PA (US); Rajendra Pradhan, New Haven, CT (US); Douglas L. Sheridan, Branford, CT (US); Marc Vallee, Lexington, MA (US); Yang Dai, Lexington, MA (US); Xiang Gao, Guilford, CT (US)

(73) Assignee: Alexion Pharmaceuticals, Inc., Boston, MA (US)

(21) Appl. No.: 17/289,150

(22) PCT Filed: Oct. 30, 2019
(86) PCT No.: PCT/US2019/058842

§ 371 Date: Apr. 27, 2021

Related U.S. Application Data
(60) Provisional application No. 62/752,570, filed on Oct. 30, 2018.

(51) Int. Cl. C07K 16/18; A61K 38/47; A61P 7/00

(52) U.S. Cl. CPC C07K 16/18; A61K 38/47; A61K 2039/505; C12Y 302/01036; A61P 7/00

(57) ABSTRACT

Provided herein are compositions and methods for treating a human patient with a complement-associated condition (e.g., PNH or aHUS) by subcutaneously co-administering to the patient a hyaluronidase (e.g., rHuPH20) and an anti-C5 antibody, or antigen binding fragment thereof (e.g., ravulizumab).

### RELATED APPLICATIONS

This application claims priority to, and the benefit of, U.S. Provisional Application No. 62/752,570 (filed on Oct. 30, 2018), the entire contents which are incorporated herein by reference.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

While a properly functioning complement system provides a robust defense against infecting microbes, inappropriate regulation or activation of the complement pathways has been implicated in the pathogenesis of a variety of disorders, including paroxysmal nocturnal hemoglobinuria (PNH) and atypical hemolytic uremic syndrome (aHUS). PNH and aHUS, are both ultra-rare disorders driven by chronic uncontrolled complement activation. The resulting inflammation and cellular damage lead to the devastating clinical manifestations of these diseases.

PNH is a condition in which uncontrolled complement activity leads to systemic complications, principally through intravascular hemolysis and platelet activation (see Socié G, et al., French Society of Haematology. Lancet. 1996; 348(9027):573-577 and Brodsky, R., Blood. 2014; 124(18):2804-2811). Persistent intravascular hemolysis may be triggered by various stressors, such as infection or physical exertion, and this leads to smooth muscle contraction (free hemoglobin), chronic anemia, and an increased risk of severe thromboembolism. Thromboembolism is the most common cause of mortality in patients with PNH, and pulmonary hypertension and end-organ damage of vital organs, such as the liver, kidneys, brain, and intestines, are sequelae of such events (Hillmen, P., et al, Am. J. Hematol. 2010; 85(8):553-559). Due to these adverse pathologic processes, patients with PNH have a decreased quality of life (QoL), which may include debilitating fatigue, chronic pain, poor physical function, shortness of breath, abdominal pain, erectile dysfunction, a need for anticoagulation, blood transfusions and in some instances, need for dialysis (Weitz, I C., et al., Thromb Res. 2012; 130(3):361-368).

Hemolytic uremic syndrome (HUS) is characterized by thrombocytopenia, microangiopathic hemolytic anemia, and acute renal failure. HUS is classified as one of two types: diarrheal-associated (D+ HUS; also referred to as shiga toxin producing E. coli (STEC)-HUS or typical HUS) and non-diarrheal or atypical HUS (aHUS). D+ HUS is the most common form, accounting for greater than 90% of cases and is caused by a preceding illness with a shiga-like toxin-producing bacterium, e.g., E. coli O157:H7.

aHUS can be genetic, acquired, or idiopathic. Hereditable forms of aHUS can be associated with mutations in a number of human complement components including, e.g., complement factor H (CFH), membrane cofactor protein (MCP), complement factor I (CFI), C4b-binding protein (C4BP), complement factor B (CFB), and complement component 3 (C3). See, e.g., Caprioli et al. (2006) Blood 108:1267-1279. Certain mutations in the gene encoding CD55, though not yet implicated in aHUS, are associated with the severity of aHUS. See, e.g., Esparza-Gordillo et al. (2005) Hum Mol Genet 14:703-712.

aHUS is rare and has a mortality rate of up to 25%. Many patients with this disease will sustain permanent neurological or renal impairment, e.g., at least 50% of aHUS patients progress to end-stage renal failure (ESRF). See, e.g., Kavanagh et al. (2006) British Medical Bulletin 77 and 78:5-22. Until recently, treatment options for patients with aHUS were limited and often involved plasma infusion or plasma exchange. In some cases, aHUS patients undergo uni- or bilateral nephrectomy or renal transplantation (see Artz et al. (2003) Transplantation 76:821-826). However, recurrence of the disease in treated patients is common.

Patients with PNH or aHUS are at risk of substantial morbidity and mortality. Accordingly, it is an object of the present invention to provide improved methods for treating patients with PNH or aHUS.

### SUMMARY

Provided herein are compositions and methods for treating a human patient with a complement-associated condition (e.g., PNH or aHUS) by subcutaneously co-administering to the patient a hyaluronidase (e.g., a recombinant human hyaluronidase) and an anti-C5 antibody, or antigen binding fragment thereof. Co-administration of a hyaluronidase (e.g., a recombinant human hyaluronidase) and an anti-C5 antibody, or antigen binding fragment thereof, facilitates a larger amount of the anti-C5 antibody, or antigen binding fragment thereof, to be administered in a single time, thereby allowing less frequent dosing. In addition, this particular combination is particularly advantageous in that it provides patients with a self-administered dosing option that eliminates the patient burden associated with intravenous (IV) infusions (e.g., loss of work time, disruption of routine associated with dosing frequency, and prolonged infusion times).

Any suitable hyaluronidase (e.g., a recombinant human hyaluronidase) can be used in the methods described herein, including, but not limited to, those described in U.S. Pat. No. 7,767,429 (e.g., SEQ ID NO:1), U.S. Pat. No. 7,846,431 (e.g., SEQ ID NO:1), U.S. Pat. No. 7,871,607 (e.g., SEQ ID NO:1), U.S. Pat. No. 8,105,586 (e.g., SEQ ID NO:1), U.S. Pat. No. 8,202,517 (e.g., SEQ ID NO:1), U.S. Pat. No. 8,257,699 (e.g., SEQ ID NO:1), U.S. Pat. No. 8,450,470 (e.g., SEQ ID NO:1), U.S. Pat. No. 8,431,124 (e.g., SEQ ID NO:1), U.S. Pat. No. 8,431,380 (e.g., SEQ ID NO:1), U.S. Pat. No. 8,580,252 (e.g., SEQ ID NO:1), U.S. Pat. No. 8,765,685 (e.g., SEQ ID NO:1), U.S. Pat. No. 8,772,246 (e.g., SEQ ID NO:1), U.S. Pat. No. 9,211,315 (e.g., SEQ ID NO:1), U.S. Pat. No. 9,562,223 (e.g., SEQ ID NO:1), U.S. Pat. No. 9,677,061 (e.g., SEQ ID NO:1), U.S. Pat. No. 9,677,062 (e.g., SEQ ID NO:1), and U.S. Pat. No. 5,721,348 (e.g., SEQ ID NO:6), the contents of each of which is expressly incorporated herein by reference. The generation of such recombinant human hyaluronidases are described in U.S. Pat. Nos. 7,767,429, 7,871,607 and US20060104968, the contents of each of which is expressly incorporated herein by reference.

An exemplary recombinant human hyaluronidase is rHuPH20, i.e., the active ingredient in the commercial product Hylenex® recombinant (hyaluronidase human injection), which is supplied as ENHANZE® drug product.

In one embodiment, the recombinant human hyaluronidase includes a sequence of amino acids in any one of SEQ ID NOs:51-60, or has at least 80%, 81%, 82%, 83%, 84%, 85%, 86%, 87%, 88%, 89%, 90%, 91%, 92%, 93%, 94%, 95%, 95%, 97%, 98%, or 99% sequence identity to a sequence of amino acids included in SEQ ID NO:51-60 and retains hyaluronidase activity. In another embodiment, the recombinant human hyaluronidase comprises the amino acid sequence set forth in SEQ ID NO:51. In another embodiment, the recombinant human hyaluronidase consists of the amino acid sequence set forth in SEQ ID NO:51. In another embodiment, the recombinant human hyaluronidase comprises the amino acid sequence set forth in SEQ ID NO:52. In another embodiment, the recombinant human hyaluronidase consists of the amino acid sequence set forth in SEQ ID NO:52. In another embodiment, the recombinant human hyaluronidase comprises the amino acid sequence set forth in SEQ ID NO:53. In another embodiment, the recombinant human hyaluronidase consists of the amino acid sequence set forth in SEQ ID NO:53. In another embodiment, the recombinant human hyaluronidase comprises the amino acid sequence set forth in SEQ ID NO:54. In another embodiment, the recombinant human hyaluronidase consists of the amino acid sequence set forth in SEQ ID NO:54. In another embodiment, the recombinant human hyaluronidase comprises the amino acid sequence set forth in SEQ ID NO:55. In another embodiment, the recombinant human hyaluronidase consists of the amino acid sequence set forth in SEQ ID NO:55. In another embodiment, the recombinant human hyaluronidase comprises the amino acid sequence set forth in SEQ ID NO:56. In another embodiment, the recombinant human hyaluronidase consists of the amino acid sequence set forth in SEQ ID NO:56. In another embodiment, the recombinant human hyaluronidase comprises the amino acid sequence set forth in SEQ ID NO:57. In another embodiment, the recombinant human hyaluronidase consists of the amino acid sequence set forth in SEQ ID NO:57. In another embodiment, the recombinant human hyaluronidase comprises the amino acid sequence set forth in SEQ ID NO:58. In another embodiment, the recombinant human hyaluronidase consists of the amino acid sequence set forth in SEQ ID NO:58. In another embodiment, the recombinant human hyaluronidase comprises the amino acid sequence set forth in SEQ ID NO:59. In another embodiment, the recombinant human hyaluronidase consists of the amino acid sequence set forth in SEQ ID NO:59. In another embodiment, the recombinant human hyaluronidase comprises the amino acid sequence set forth in SEQ ID NO:60. In another embodiment, the recombinant human hyaluronidase consists of the amino acid sequence set forth in SEQ ID NO:60.

In another embodiment, the recombinant human hyaluronidase is rHuPH20 administered in a formulation comprising approximately 110 kU/mL of rHuPH20, 130 mM sodium chloride, 10 mM L-Histidine/hydrochloride, 10 mM L-Methionine and 0.2% w/w polysorbate 80.

Any suitable anti-C5 antibody, or antigen binding fragment thereof, can be used in the methods described herein. An exemplary anti-C5 antibody is ravulizumab (also known as ALXN1210 and antibody BNJ441) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2, and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively.

In another embodiment, the antibody comprises VH and VL regions having the amino acid sequences set forth in SEQ ID NO: 12 and SEQ ID NO: 8, respectively. In another embodiment, the antibody comprises a heavy chain constant region as set forth in SEQ ID NO:13. In another embodiment, the antibody comprises a heavy chain polypeptide as set forth in SEQ ID NO:14 and a light chain polypeptide as set forth in SEQ ID NO:11. In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 6.0 and 25° C. with a K_{D}≥10 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

In another embodiment, the anti-C5 antibody is ravulizumab administered in a formulation comprising 1100 mg of ravulizumab, 50 mM sodium phosphate, 25 mM arginine, 5% sucrose, and 0.05% polysorbate 80.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region of the 8110 antibody having the sequence set forth in SEQ ID NO: 35, and the VL region of the 8110 antibody having the sequence set forth in SEQ ID NO: 36.

Another exemplary anti-C5 antibody is the 305LO5 antibody described in US2016/0176954A1. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region of the 305L05 antibody having the sequence set forth in SEQ ID NO: 43, and the VL region of the 305L05 antibody having the sequence set forth in SEQ ID NO: 44.

Another exemplary anti-C5 antibody is the SKY59 antibody described in Fukuzawa T., et al., Rep. 2017 Apr. 24; 7(1):1080). In one embodiment, the antibody comprises the heavy and light chain CDRs or variable regions of the SKY59 antibody. In another embodiment, the antibody, or antigen binding fragment thereof, comprises a heavy chain comprising SEQ ID NO: 45 and a light chain comprising SEQ ID NO: 46.

Another exemplary anti-C5 antibody is the REGN3918 antibody (also known as H4H12166PP) described in US20170355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

In another embodiment, the antibody competes for binding with, and/or binds to the same epitope on C5 as, the above-mentioned antibodies (e.g., eculizumab, ravulizumab, 7086 antibody, 8110 antibody, 305LO5 antibody, SKY59 antibody, or REGN3918 antibody). In another embodiment, the antibody has at least about 90% variable region amino acid sequence identity with the above-mentioned antibodies (e.g., at least about 90%, 91%, 92%, 93%, 94%, 95%, 96%, 97%, 98% or 99% variable region identity).

In one embodiment, the hyaluronidase (e.g., a recombinant human hyaluronidase) and antibody, or antigen-binding fragment thereof, are administered simultaneously in separate formulations. In another embodiment, the hyaluronidase (e.g., a recombinant human hyaluronidase) and anti-C5 antibody, or antigen-binding fragment thereof, are administered sequentially (e.g., as separate formulations). For example, the hyaluronidase (e.g., a recombinant human hyaluronidase) can be administered first followed by (e.g., immediately followed by) the administration of the anti-C5 antibody, or antigen-binding fragment thereof, or vice versa. Such concurrent or sequential administration preferably results in both the hyaluronidase (e.g., a recombinant human hyaluronidase) and anti-C5 antibody, or antigen-binding fragment thereof, being simultaneously present in treated patients.

In another embodiment, the hyaluronidase (e.g., a recombinant human hyaluronidase) and antibody, or antigen-binding fragment thereof, are administered simultaneously in a single formulation. For example, the hyaluronidase (e.g., a recombinant human hyaluronidase) and antibody, or antigen-binding fragment thereof, can be mixed and co-administered in a single formulation.

In one embodiment, the hyaluronidase is a recombinant human hyaluronidase, for example, rHuPH20 (ENHANZE®) administered at a concentration of 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000, 30,500, 31,000, 31,500, 32,000, 32,500, 33,000, 33,500, 34,000, 34,500, 35,000, 35,500, 36,000, 36,500, 37,000, 37,500, 38,000, 38, 500, 39,000, 39,500, 40,000, 40,500, 45,000, or 50,000 units. In a particular embodiment, rHuPH20 is administered at a concentration of 10,000 units. In another particular embodiment, rHuPH20 is administered at a concentration of 20,000 units. In another particular embodiment, rHuPH20 is administered at a concentration of 40,000 units.

In another embodiment, the antibody, or antigen-binding fragment thereof, is ravulizumab administered at a dose of 500 mg, 600 mg, 700 mg, 800 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, 5,000 mg, 5,100 mg, 5,200 mg, 5,300 mg, 5,400 mg, 5,500 mg, 5,600 mg, 5,700 mg, 5,800 mg, 5,900 mg, 6,000 mg, or 7,000 mg. In a particular embodiment, the antibody, or antigen-binding fragment thereof, is ravulizumab administered at a dose of 500 mg. In another particular embodiment, the antibody, or antigen-binding fragment thereof, is ravulizumab administered at a dose of 1000 mg. In another particular embodiment, the antibody, or antigen-binding fragment thereof, is ravulizumab administered at a dose of 2000 mg.

In another embodiment, the antibody, or antigen-binding fragment thereof, is ravulizumab and the hyaluronidase is rHuPH20, and the patient is separately administered ravulizumab at 500 mg and 10,000 units of rHuPH20 (e.g., sequentially or simultaneously as separate formulations). In one embodiment, rHuPH20 is administered just prior to administration of ravulizumab. In another embodiment, ravulizumab is administered just prior to administration of rHuPH20.

In another embodiment, the antibody, or antigen-binding fragment thereof, is ravulizumab and the hyaluronidase is rHuPH20, and the patient is administered a single formulation comprising ravulizumab at 500 mg and 10,000 units of rHuPH20.

In another embodiment, the antibody, or antigen-binding fragment thereof, is ravulizumab and the hyaluronidase is rHuPH20, and the patient is separately administered ravulizumab at 1000 mg and 20,000 units of rHuPH20 (e.g., sequentially or simultaneously as separate formulations). In one embodiment, rHuPH20 is administered just prior to administration of ravulizumab. In another embodiment, ravulizumab is administered just prior to administration of rHuPH20.

In another embodiment, the antibody, or antigen-binding fragment thereof, is ravulizumab and the hyaluronidase is rHuPH20, and the patient is administered a single formulation comprising ravulizumab at 1000 mg and 20,000 units of rHuPH20.

In another embodiment, the antibody, or antigen-binding fragment thereof, is ravulizumab and the hyaluronidase is rHuPH20, and the patient is separately administered ravulizumab at 2000 mg and 40,000 units of rHuPH20 (e.g., sequentially or simultaneously as separate formulations). In one embodiment, rHuPH20 is administered just prior to administration of ravulizumab. In another embodiment, ravulizumab is administered just prior to administration of rHuPH20.

In another embodiment, the antibody, or antigen-binding fragment thereof, is ravulizumab and the hyaluronidase is rHuPH20, and the patient is administered a single formulation comprising ravulizumab at 2000 mg and 40,000 units of rHuPH20.

In another embodiment, the hyaluronidase (e.g., a recombinant human hyaluronidase) and anti-C5 antibody, or antigen binding fragment thereof, are administered to the patient once every two weeks, once every three weeks, once a month, once every month and a half, once every two months, or once every three months.

Subcutaneous administration of a hyaluronidase (e.g., rHuPH20) and an anti-C5 antibody, or antigen binding fragment thereof, (e.g., ravulizumab) according to the methods described herein can be accomplished by any suitable means. In addition, the hyaluronidase (e.g., a recombinant human hyaluronidase) and anti-C5 antibody, or antigen binding fragment thereof, can be administered subcutaneously by a medical professional or self-administered. In one embodiment, the hyaluronidase (e.g., a recombinant human hyaluronidase) and antibody, or antigen-binding fragment thereof, are subcutaneously administered to the patient via an infusion pump. In another embodiment, the hyaluronidase (e.g., a recombinant human hyaluronidase) and anti-C5 antibody, or antigen binding fragment thereof, is administered subcutaneously using an on-body delivery system (OBDS).

In some embodiments, the patients treated according to the methods described herein have been vaccinated against meningococcal infections prior to initiating treatment. In one embodiment, patients treated according to the methods described herein are vaccinated against meningococcal serotypes A, C, Y, W135, and/or B. In another embodiment, patients treated according to the methods described herein receive the MCV4 vaccination at least 56 days prior to dosing with the anti-C5 antibody, or antigen binding fragment thereof (e.g., ravulizumab). In one embodiment, where a patient has not already been vaccinated for serotype B meningococcal infections, the patient is vaccinated for serotype B meningococcal infections at least 56 days prior to initiating treatment, with a booster administered at least 28 days prior to initiating treatment.

In some embodiments, the patient is administered one or more additional therapeutic agents prior to and/or during treatment. For example, in one embodiment, the patient is administered an antibiotic (e.g., 500 mg of penicillin orally twice daily or ciprofloxacin) prior to and/or during treatment. In another embodiment, the hyaluronidase (e.g., a recombinant human hyaluronidase) and anti-C5 antibody, or antigen binding fragment thereof, are administered in combination with no more than three additional agents. In another embodiment, the hyaluronidase (e.g., a recombinant human hyaluronidase) and anti-C5 antibody, or antigen binding fragment thereof, are administered in combination with no more than two additional agents. In another embodiment, the hyaluronidase (e.g., a recombinant human hyaluronidase) and anti-C5 antibody, or antigen binding fragment thereof, are administered in combination with no more than one additional agent. In another embodiment, no additional agents are administered in combination with the hyaluronidase (e.g., a recombinant human hyaluronidase) and anti-C5 antibody, or antigen binding fragment thereof.

In another aspect, the treatment regimens described herein are sufficient to maintain particular serum trough concentrations of the anti-C5 antibody, or antigen binding fragment thereof. For example, in one embodiment, the treatment maintains a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, or 400 μg/ml or greater. In another embodiment, the treatment maintains a serum trough concentration of the anti-C5 antibody, or antigen binding fragment thereof, of 100 μg/ml or greater. In another embodiment, the treatment maintains a serum trough concentration of the anti-C5 antibody, or antigen binding fragment thereof, of 150 μg/ml or greater. In another embodiment, the treatment maintains a serum trough concentration of the anti-C5 antibody, or antigen binding fragment thereof, of 200 μg/ml or greater. In another embodiment, the treatment maintains a serum trough concentration of the anti-C5 antibody, or antigen binding fragment thereof, of 250 μg/ml or greater. In another embodiment, the treatment maintains a serum trough concentration of the anti-C5 antibody, or antigen binding fragment thereof, of 300 μg/ml or greater. In another embodiment, the treatment maintains a serum trough concentration of the anti-C5 antibody, or antigen binding fragment thereof, of between 100 μg/ml and 200 μg/ml. In another embodiment, the treatment maintains a serum trough concentration of the anti-C5 antibody, or antigen binding fragment thereof, of about 175 μg/ml.

In some embodiments, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

The efficacy of the treatment methods provided herein can be assessed using any suitable means. In one embodiment, the methods described herein result in the amelioration of at least one symptom of the complement-associated disease. For example, in the context of PNH, the treatment may alleviate of one more symptoms selected from the group consisting of fatigue, abdominal pain, dyspnea, dysphagia, chest pain, and/or erectile dysfunction). In the context of aHUS, for example, the treatment may alleviate one or more symptoms selected from the group consisting of severe hypertension, proteinuria, uremia, lethargy/fatigue, irritability, thrombocytopenia, microangiopathic hemolytic anemia, and/or renal function impairment (e.g., acute renal failure).

In another embodiment, the treatment results in terminal complement inhibition.

In another embodiment, the treatment produces a reduction in the need for blood transfusions.

In another embodiment, the treatment produces an increase in hemoglobin stabilization from the patient's pre-treatment baseline.

In another embodiment, the treatment produces a shift toward normal levels of a hemolysis-related hematologic biomarker selected from the group consisting of free hemoglobin, haptoglobin, reticulocyte count, PNH red blood cell (RBC) clone and D-dimer.

In another embodiment, the treatment produces a reduction in major adverse vascular events (MAVEs). In another embodiment, the treatment produces a shift toward normal levels of a chronic disease associated biomarker selected from the group consisting estimated glomerular filtration rate (eGFR) and spot urine:albumin:creatinine and plasma brain natriuretic peptide (BNP).

In another embodiment, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale.

In a particular embodiment, lactate dehydrogenase (LDH) levels are used to evaluate responsiveness to a therapy (e.g., a reduction of hemolysis as assessed by lactate dehydrogenase (LDH) levels is indicative of an improvement in at least one sign of PNH). For example, in one embodiment, the treatments described herein result in a normalization of LDH levels.

In one embodiment, patients treated according to the disclosed methods experience reductions in LDH levels to within normal levels or to within 10%, 20%, 30%, 40% or within 50% below what is considered the upper limit of normal level (e.g., within 105-333 IU/L (international units per liter). In another embodiment, the patient's LDH levels are normalized throughout maintenance period of treatment. In another embodiment, the treated patient's LDH levels are normalized at least at least 95% of the time while on the maintenance period of treatment. In another embodiment, the treated patient's LDH levels are normalized at least at least 90%, 85% or 80% of the time while on the maintenance period of treatment.

Exemplary complement-associated conditions that can be treated according to the methods described herein include, but are not limited to, rheumatoid arthritis, antiphospholipid antibody syndrome, lupus nephritis, ischemia-reperfusion injury, atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome. In a particular embodiment, the complement-associated condition is atypical hemolytic uremic syndrome (aHUS). In another particular embodiment, the complement-associated condition is paroxysmal nocturnal hemoglobinuria (PNH).

Also provided are kits that include an anti-C5 antibody, or antigen binding fragment thereof (e.g., ravulizumab), and a hyaluronidase (e.g., rHuPH20), in therapeutically effective amounts adapted for use in the methods described herein. For example, in one embodiment, a kit for treating a complement-associated condition in a human patient is provided, the kit comprising: (a) a dose of an anti-C5 antibody, or antigen binding fragment thereof, (b) a dose of hyaluronidase (e.g., a recombinant human hyaluronidase), and (c) instructions for using the anti-C5 antibody, or antigen binding fragment thereof, and hyaluronidase (e.g., a recombinant human hyaluronidase) in the method of any one of the preceding claims. In one embodiment, the anti-C5 antibody is ravulizumab. In another embodiment, the recombinant human hyaluronidase is rHuPH20 (ENHANZE®).

Further provided is a device comprising a prefilled cartridge of ravulizumab and rHuPH20 for subcutaneous administration co-packaged with an on-body injector. In one embodiment, the device is sterile, for single use, disposable, and/or electro-mechanical.

### BRIEF DESCRIPTION OF THE DRAWING

FIG. 1 is a schematic depicting the study design. Subjects are randomly assigned to Cohort 1 or Cohort 2, randomly assigned to Cohort 3 or Cohort 5, and sequentially assigned to Cohort 4. ^{a}Dosing is staggered, but the end of study for each subject is Day 200 or the time point at which complement activity has normalized, if later than Day 200. ^{b}For Cohorts 1 through 4, a sentinel dosing approach is used (i.e., 2 subjects in a cohort with 12 subjects and 1 subject in a cohort with 6 subjects are dosed prior to dosing the remaining subjects within the cohort).

### DETAILED DESCRIPTION

### I. Hyaluronidases

As used herein, a hyaluronan degrading enzyme refers to an enzyme that catalyzes the cleavage of a hyaluronan polymer (also referred to as hyaluronic acid or HA) into smaller molecular weight fragments. Exemplary of hyaluronan degrading enzymes are hyaluronidases, and particular chondroitinases and lyases that have the ability to depolymerize hyaluronan. Exemplary chondroitinases that are hyaluronan degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase comprises two enzymes, chondroitin-sulfate-ABC endolyase (EC 4.2.2.20) and chondroitin-sulfate-ABC exolyase (EC 4.2.2.21). An exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO:98; Sato et al. (1994) Appl. Microbiol. Biotechnol. 41(1):39-46). Exemplary chondroitinase AC enzymes from the bacteria include, but are not limited to, those from Flavobacterium heparinum Victivallis vadensis, set forth in SEQ ID NO:99, and Arthrobacter aurescens (Tkalec et al. (2000) Applied and Environmental Microbiology 66(1):29-35; Ernst et al. (1995) Critical Reviews in Biochemistry and Molecular Biology 30(5):387-444). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133).

As used herein, hyaluronidase refers to a class of hyaluronan degrading enzymes. Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include, hyaluronidases from cows (SEQ ID NOS:10, 11, 64 of U.S. Pat. No. 8,568,713) and BH55 (U.S. Pat. Nos. 5,747,027 and 5,827,721), yellow jacket wasp (SEQ ID NOS:12 and 13 of U.S. Pat. No. 8,568,713), honey bee (SEQ ID NO:14 of U.S. Pat. No. 8,568,713), white-face hornet (SEQ ID NO:15 of U.S. Pat. No. 8,568,713), paper wasp (SEQ ID NO:16 of U.S. Pat. No. 8,568,713), mouse (SEQ ID NOS:17-19, and 32 of U.S. Pat. No. 8,568,713), pig (SEQ ID NOS:20-21 of U.S. Pat. No. 8,568,713), rat (SEQ ID NOS:22-24, and 31 of U.S. Pat. No. 8,568,713), rabbit (SEQ ID NO:25 of U.S. Pat. No. 8,568,713), sheep (SEQ ID NOS:26, 27, 63 and 65 of U.S. Pat. No. 8,568,713), orangutan (SEQ ID NO:28 of U.S. Pat. No. 8,568,713), cynomolgus monkey (SEQ ID NO:29 of U.S. Pat. No. 8,568,713), guinea pig (SEQ ID NO:30 of U.S. Pat. No. 8,568,713), Arthrobacter sp. (strain FB24) (SEQ ID NO:67 of U.S. Pat. No. 8,568,713), Bdellovibrio bacteriovorus (SEQ ID NO:68 of U.S. Pat. No. 8,568,713), Propionibacterium acnes (SEQ ID NO:69 of U.S. Pat. No. 8,568,713), Streptococcus agalactiae ((SEQ ID NO:70 of U.S. Pat. No. 8,568,713); 18RS21 (SEQ ID NO:71 of U.S. Pat. No. 8,568,713); serotype Ia (SEQ ID NO:72 of U.S. Pat. No. 8,568,713); serotype III (SEQ ID NO:73 of U.S. Pat. No. 8,568,713), Staphylococcus aureus (strain COL) (SEQ ID NO:74 of U.S. Pat. No. 8,568,713); strain MRSA252 (SEQ ID NOS:75 and 76 of U.S. Pat. Nos. 8,568,713 and 8,568,713); strain MSSA476 (SEQ ID NO:77 of U.S. Pat. No. 8,568,713); strain NCTC 8325 (SEQ ID NO:78 of U.S. Pat. No. 8,568,713); strain bovine RF122 (SEQ ID NOS:79 and 80 of U.S. Pat. No. 8,568,713); strain USA300 (SEQ ID NO:81 of U.S. Pat. No. 8,568,713), Streptococcus pneumoniae (SEQ ID NO:82 of U.S. Pat. No. 8,568,713); strain ATCC BAA-255/R6 (SEQ ID NO:83 of U.S. Pat. No. 8,568,713); serotype 2, strain D39/NCTC 7466 (SEQ ID NO:84 of U.S. Pat. No. 8,568,713), Streptococcus pyogenes (serotype (SEQ ID NO:85 of U.S. Pat. No. 8,568,713); serotype M2, strain MGAS10270 (SEQ ID NO:86 of U.S. Pat. No. 8,568,713); serotype M4, strain MGAS10750 (SEQ ID NO:87 of U.S. Pat. No. 8,568,713); serotype M6 (SEQ ID NO:88 of U.S. Pat. No. 8,568,713); serotype M12, strain MGAS2096 (SEQ ID NOS:89 and 90 of U.S. Pat. No. 8,568,713); serotype M12, strain MGAS9429 (SEQ ID NO:91 of U.S. Pat. No. 8,568,713); serotype M28 (SEQ ID NO:92 of U.S. Pat. No. 8,568,713); Streptococcus suis (SEQ ID NOS:93-95 of U.S. Pat. No. 8,568,713); Vibrio fischeri (strain ATCC 700601/ES114 (SEQ ID NO:96 of U.S. Pat. No. 8,568,713), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607). Hyaluronidases also include those of human origin. Exemplary human hyaluronidases include PH20 (SEQ ID NO:51), HYAL1 (SEQ ID NO:36 of U.S. Pat. No. 8,568,713), HYAL2 (SEQ ID NO:37 of U.S. Pat. No. 8,568,713), HYAL3 (SEQ ID NO:38 of U.S. Pat. No. 8,568,713), and HYAL4 (SEQ ID NO:36 of U.S. Pat. No. 8,568,713). The sequences and contents of U.S. Pat. No. 8,568,713 are expressly incorporated herein by reference. Also included amongst hyaluronidases are soluble hyaluronidases, including, ovine and bovine PH20, soluble human PH20 and soluble rHuPH20. Examples of commercially available bovine or ovine soluble hyaluronidases Vitrase® (ovine hyaluronidase) and Amphadase® (bovine hyaluronidase).

Hyaluronidases as described herein include precursor hyaluronan degrading enzyme polypeptides and mature hyaluronan degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptides set forth in SEQ ID NO:51 or the mature form thereof set forth in SEQ ID NO:52. Hyaluronidases also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art.

As used herein, a soluble hyaluronidase refers to a polypeptide characterized by its solubility under physiologic conditions. Soluble hyaluronidases can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, hyaluronidase activity refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as soluble rHuPH20, are known in the art. Exemplary assays include the microturbidity assay described below (see e.g., Example 3 of U.S. Pat. No. 8,568,713) that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

As used herein, “functionally equivalent amount” or grammatical variations thereof, with reference to a hyaluronan degrading enzyme, refers to the amount of hyaluronan degrading enzyme that achieves the same effect as an amount (such as a known number of Units of hyaluronidase activity) of a reference enzyme, such as a hyaluronidase. For example, the activity of any hyaluronan degrading enzyme can be compared to the activity of rHuPH20 to determine the functionally equivalent amount of a hyaluronan degrading enzyme that would achieve the same effect as a known amount of rHuPH20. For example, the ability of a hyaluronan degrading enzyme to act as a spreading or diffusing agent can be assessed by injecting it into the lateral skin of mice with trypan blue (see e.g. U.S. Pat. Publication No. 20050260186), and the amount of hyaluronan degrading enzyme required to achieve the same amount of diffusion as, for example, 100 units of a Hyaluronidase Reference Standard, can be determined. The amount of hyaluronan degrading enzyme required is, therefore, functionally equivalent to 100 units.

Exemplary hyaluronan degrading enzymes are hyaluronidases, particularly soluble hyaluronidases, such as a PH20, or a truncated form thereof. The PH20 can be, for example, an ovine, bovine or truncated human PH20. The human PH20 mRNA transcript is normally translated to generate a 509 amino acid precursor polypeptide (SEQ ID NO:51; and replicated below) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 is, therefore, a 474 amino acid polypeptide set forth in SEQ ID NO:52. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:1. Thus, a 474 amino acid GPI-anchored mature polypeptide with an amino acid sequence set forth in SEQ ID NO:52 is produced.

The amino acid sequence of the human PH20 precursor polypeptide (SEQ ID NO:51; 509 amino acids) is as follows:

MGVLKFKHIFFRSFVKSSGVSQIVFTFLLIPCCLTLNFRAPPVIPNVPFL
WAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTEATEKAKQEFEKAGKDFL
VETIKLGKLLRPNHLWGYYLFPDCYNHHYKKPGYNGSCFNVEIKRNDDLS
WLWNESTALYPSIYLNTQQSPVAATLYVRNRVREAIRVSKIPDAKSPLPV
FAYTRIVFTDQVLKFLSQDELVYTFGETVALGASGIVIWGTLSIMRSMKS
CLLLDNYMETILNPYIINVTLAAKMCSQVLCQEQGVCIRKNWNSSDYLHL
NPDNFAIQLEKGGKFTVRGKPTLEDLEQFSEKFYCSCYSTLSCKEKADVK
DTDAVDVCIADGVCIDAFLKPPMETEEPQIFYNASPSTLSATMFIVSILF
LIISSVASL.

Human PH20 exhibits hyaluronidase activity at both neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at both neutral and acid pH. It appears that PH20 contains two catalytic sites at distinct regions of the polypeptide: the Peptide 1 and Peptide 3 regions (Cherr et al., (2001) Matrix Biology 20:515-525). Evidence suggests that the Peptide 1 region of PH20, which corresponds to amino acid positions 107-137 of the mature polypeptide set forth in SEQ ID NO:52 and positions 142-172 of the precursor polypeptide set forth in SEQ ID NO:51, is required for enzyme activity at neutral pH. Amino acids at positions 111 and 113 (corresponding to the mature PH20 polypeptide set forth in SEQ ID NO:52) within this region appear to be important for activity, as mutagenesis by amino acid replacement results in PH20 polypeptides with 3% hyaluronidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:52, and positions 277-297 of the precursor polypeptide set forth in SEQ ID NO:51, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247:810-814).

In addition to the catalytic sites, PH20 also contains a hyaluronan-binding site. Experimental evidence suggest that this site is located in the Peptide 2 region, which corresponds to amino acid positions 205-235 of the precursor polypeptide set forth in SEQ ID NO: 51 and positions 170-200 of the mature polypeptide set forth in SEQ ID NO:52. This region is highly conserved among hyaluronidases and is similar to the heparin binding motif. Mutation of the arginine residue at position 176 (corresponding to the mature PH20 polypeptide set forth in SEQ ID NO:52) to a glycine results in a polypeptide with only about 1% of the hyaluronidase activity of the wild type polypeptide (Arming et al., (1997) Eur. J. Biochem. 247:810-814).

There are seven potential N-linked glycosylation sites in human PH20 at N82, N166, N235, N254, N368, N393, N490 of the polypeptide exemplified in SEQ ID NO:51. Because amino acids 36 to 464 of SEQ ID NO:51 appears to contain the minimally active human PH20 hyaluronidase domain, the N-linked glycosylation site N-490 is not required for proper hyaluronidase activity. There are six disulfide bonds in human PH20. Two disulphide bonds between the cysteine residues C60 and C351 and between C224 and C238 of the polypeptide exemplified in SEQ ID NO:51 (corresponding to residues C25 and C316, and C189 and C203 of the mature polypeptide set forth in SEQ ID NO:52, respectively). A further four disulphide bonds are formed between the cysteine residues C376 and C387; between C381 and C435; between C437 and C443; and between C458 and C464 of the polypeptide exemplified in SEQ ID NO: 51 (corresponding to residues C341 and C352; between C346 and C400; between C402 and C408; and between C423 and C429 of the mature polypeptide set forth in SEQ ID NO:52, respectively).

As used herein, soluble recombinant human PH20 (rHuPH20) refers to a soluble form of human PH20 that is recombinantly expressed in Chinese Hamster Ovary (CHO) cells. Soluble human PH20 or sHuPH20 includes mature polypeptides lacking all or a portion of the glycosylphospatidylinositol (GPI) attachment site at the C-terminus such that upon expression, the polypeptides are soluble.

Soluble rHuPH20 is encoded by nucleic acid that includes the signal sequence and is set forth in SEQ ID NO:61. Also included are DNA molecules that are allelic variants thereof and other soluble variants. The nucleic acid encoding soluble rHuPH20 is expressed in CHO cells which secrete the mature polypeptide. As produced in the culture medium, there is heterogeneity at the C-terminus so that the product includes a mixture of species that can include any one or more of SEQ ID NOs:53-60 in various abundance. Accordingly, exemplary sHuPH20 polypeptides include mature polypeptides having an amino acid sequence set forth in any one of SEQ ID NOS:53-60. Other variants can have 60%, 70%, 80%, 85%, 90%, 91%, 92%, 93%, 94%, 95%, 96%, 97%, 98%, 99% or more sequence identity with any of SEQ ID NOS:53-60 as long they retain a hyaluronidase activity and are soluble. Corresponding allelic variants and other variants also are included, including those corresponding to the precursor human PH20 polypeptides set forth in SEQ ID NOS:51.

rHuPH20 was approved by the US Food and Drug Administration in 2005 as an adjuvant to increase the dispersion and absorption of other injected drugs. Recombinant human hyaluronidase PH20 is a transiently and locally-acting permeation enhancer that increases the dispersion and absorption of other injected agents. Recombinant human hyaluronidase PH20 depolymerizes hyaluronic acid (HA) at the injection site causing rapid decrease in the viscosity of the extracellular matrix, allowing bulk fluid flow and facilitating dispersion and absorption of coadministered agents (rHuPH20 Investigator's Brochure, 2018).

rHuPH20 has been injected SC immediately prior to another therapeutic agent or co-administered subcutaneously to healthy subjects and patients in 28 clinical studies conducted under the rHuPH20 US IND, including studies with single doses up to 96,000 units. No safety concerns were identified in these studies (rHuPH20 Investigator's Brochure, 2018). In addition, rHuPH20 is an excipient in 3 marketed products (Herceptin® SC, HyQvia®, and MabThera SC®) available collectively in at least 50 countries, including countries in the European Union.

rHuPH20 is a glycosylated single chain protein with up to 447 amino acids, synthesized in CHO cells. Recombinant human hyaluronidase PH20 degrades HA under physiologic conditions and acts as a spreading factor in vivo. Therefore, when combined (co-mixed) or coformulated with certain injectable drugs, rHUPH20 facilitates the absorption and dispersion of these drugs by temporarily reducing resistance to bulk fluid flow in the subcutaneous space. The permeability barrier in these tissues is restored to pre-injection levels within 24 to 48 hours after injection of rHuPH20.

Any suitable hyaluronidase (e.g., a recombinant human hyaluronidase) can be used in the methods described herein, including, but not limited to, those described in U.S. Pat. No. 7,767,429 (e.g., SEQ ID NO:1), U.S. Pat. No. 7,846,431 (e.g., SEQ ID NO:1), U.S. Pat. No. 7,871,607 (e.g., SEQ ID NO:1), U.S. Pat. No. 8,105,586 (e.g., SEQ ID NO:1), U.S. Pat. No. 8,202,517 (e.g., SEQ ID NO:1), U.S. Pat. No. 8,257,699 (e.g., SEQ ID NO:1), U.S. Pat. No. 8,450,470 (e.g., SEQ ID NO:1), U.S. Pat. No. 8,431,124 (e.g., SEQ ID NO:1), U.S. Pat. No. 8,431,380 (e.g., SEQ ID NO:1), U.S. Pat. No. 8,580,252 (e.g., SEQ ID NO:1), U.S. Pat. No. 8,765,685 (e.g., SEQ ID NO:1), U.S. Pat. No. 8,772,246 (e.g., SEQ ID NO:1), U.S. Pat. No. 9,211,315 (e.g., SEQ ID NO:1), U.S. Pat. No. 9,562,223 (e.g., SEQ ID NO:1), U.S. Pat. No. 9,677,061 (e.g., SEQ ID NO:1), U.S. Pat. No. 9,677,062 (e.g., SEQ ID NO:1), and U.S. Pat. No. 5,721,348 (e.g., SEQ ID NO:6), the contents of each of which is expressly incorporated herein by reference. The generation of such recombinant human hyaluronidases are described in U.S. Pat. Nos. 7,767,429, 7,871,607 and US20060104968, the contents of each of which is expressly incorporated herein by reference. An exemplary recombinant human hyaluronidase is rHuPH20, i.e., the active ingredient in the commercial product Hylenex® recombinant (hyaluronidase human injection), which is supplied as ENHANZE® drug product.

In one embodiment, the recombinant human hyaluronidase includes a sequence of amino acids in any one of SEQ ID NOs:51-60, or has at least 80%, 81%, 82%, 83%, 84%, 85%, 86%, 87%, 88%, 89%, 90%, 91%, 92%, 93%, 94%, 95%, 95%, 97%, 98%, or 99% sequence identity to a sequence of amino acids included in SEQ ID NO:51-60 and retains hyaluronidase activity. In another embodiment, the recombinant human hyaluronidase comprises the amino acid sequence set forth in SEQ ID NO:51. In another embodiment, the recombinant human hyaluronidase consists of the amino acid sequence set forth in SEQ ID NO:51. In another embodiment, the recombinant human hyaluronidase comprises the amino acid sequence set forth in SEQ ID NO:52. In another embodiment, the recombinant human hyaluronidase consists of the amino acid sequence set forth in SEQ ID NO:52. In another embodiment, the recombinant human hyaluronidase comprises the amino acid sequence set forth in SEQ ID NO:53. In another embodiment, the recombinant human hyaluronidase consists of the amino acid sequence set forth in SEQ ID NO:53. In another embodiment, the recombinant human hyaluronidase comprises the amino acid sequence set forth in SEQ ID NO:54. In another embodiment, the recombinant human hyaluronidase consists of the amino acid sequence set forth in SEQ ID NO:54. In another embodiment, the recombinant human hyaluronidase comprises the amino acid sequence set forth in SEQ ID NO:55. In another embodiment, the recombinant human hyaluronidase consists of the amino acid sequence set forth in SEQ ID NO:55. In another embodiment, the recombinant human hyaluronidase comprises the amino acid sequence set forth in SEQ ID NO:56. In another embodiment, the recombinant human hyaluronidase consists of the amino acid sequence set forth in SEQ ID NO:56. In another embodiment, the recombinant human hyaluronidase comprises the amino acid sequence set forth in SEQ ID NO:57. In another embodiment, the recombinant human hyaluronidase consists of the amino acid sequence set forth in SEQ ID NO:57. In another embodiment, the recombinant human hyaluronidase comprises the amino acid sequence set forth in SEQ ID NO:58. In another embodiment, the recombinant human hyaluronidase consists of the amino acid sequence set forth in SEQ ID NO:58. In another embodiment, the recombinant human hyaluronidase comprises the amino acid sequence set forth in SEQ ID NO:59. In another embodiment, the recombinant human hyaluronidase consists of the amino acid sequence set forth in SEQ ID NO:59. In another embodiment, the recombinant human hyaluronidase comprises the amino acid sequence set forth in SEQ ID NO:60. In another embodiment, the recombinant human hyaluronidase consists of the amino acid sequence set forth in SEQ ID NO:60.

In another embodiment, the recombinant human hyaluronidase is rHuPH20 administered in a formulation comprising approximately 110 kU/mL of rHuPH20, 130 mM sodium chloride, 10 mM L-Histidine/hydrochloride, 10 mM L-Methionine and 0.2% w/w polysorbate 80.

### II. Anti-C5 Antibodies

Anti-C5 antibodies (or VH/VL domains derived therefrom) suitable for use in the invention can be generated using methods well known in the art. Alternatively, art recognized anti-C5 antibodies can be used. Antibodies that compete with any of these art-recognized antibodies for binding to C5 also can be used.

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered by a protein engineering technique (e.g., by mutation, deletion, substitution, conjugation to a non-antibody moiety). For example, an antibody may include one or more variant amino acids (compared to a naturally occurring antibody), which changes a property (e.g., a functional property) of the antibody. For example, numerous such alterations are known in the art which affect, e.g., half-life, effector function, and/or immune responses to the antibody in a patient. The term antibody also includes artificial or engineered polypeptide constructs which comprise at least one antibody-derived antigen binding site.

Eculizumab (also known as Soliris®) is an anti-C5 antibody comprising heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. Eculizumab comprises a heavy chain variable region having the amino acid sequence set forth in SEQ ID NO: 7 and a light chain variable region having the amino acid sequence set forth in SEQ ID NO: 8. The variable regions of eculizumab are described in PCT/US1995/005688 and U.S. Pat. No. 6,355,245, the teachings of which are hereby incorporated by reference. Eculizumab comprises a heavy chain comprising the amino acid sequence set forth in SEQ ID NO:10 and a light chain having the amino acid sequence set forth in SEQ ID NO:11. The full heavy and light chains of eculizumab are described in PCT/US2007/006606, the teachings of which are hereby incorporated by reference.

An exemplary anti-C5 antibody is ravulizumab comprising heavy and light chains having the sequences shown in SEQ ID NOs:14 and 11, respectively, or antigen binding fragments and variants thereof. Ravulizumab (also known as BNJ441 and ALXN1210) is described in PCT/US2015/019225 and U.S. Pat. No. 9,079,949, the teachings or which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. For example, in one embodiment, the antibody comprises the CDR1, CDR2, and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

In another embodiment, the anti-C5 antibody is ravulizumab administered in a formulation comprising 1100 mg of ravulizumab, 50 mM sodium phosphate, 25 mM arginine, 5% sucrose, and 0.05% polysorbate 80.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region of the 8110 antibody having the sequence set forth in SEQ ID NO: 35, and the VL region of the 8110 antibody having the sequence set forth in SEQ ID NO: 36.

Another exemplary anti-C5 antibody is the 305LO5 antibody described in US2016/0176954A1. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region of the 305LO5 antibody having the sequence set forth in SEQ ID NO: 43, and the VL region of the 305LO5 antibody having the sequence set forth in SEQ ID NO: 44.

Another exemplary anti-C5 antibody is the SKY59 antibody described in Fukuzawa T., et al., Rep. 2017 Apr. 24; 7(1):1080). In one embodiment, the antibody comprises the heavy and light chain CDRs or variable regions of the SKY59 antibody. In another embodiment, the antibody, or antigen binding fragment thereof, comprises a heavy chain comprising SEQ ID NO: 45 and a light chain comprising SEQ ID NO: 46.

Another exemplary anti-C5 antibody is the REGN3918 antibody (also known as H4H12166PP) described in US20170355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., PCT/US2015/019225 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q311I, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434 of a native human IgG Fc constant region, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in BNJ441 (ravulizumab) and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM.

In other embodiments, the [(K_{D} of the antibody for C5 at pH 6.0 at C)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500, or 8000).

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. (see the working examples). For example, the kinetics of antibody binding to human C5 can be determined at pH 8.0, 7.4, 7.0, 6.5 and 6.0 via surface plasmon resonance (SPR) on a BIAcore 3000 instrument using an anti-Fc capture method to immobilize the antibody.

In one embodiment, the anti-C5 antibody, or antigen binding fragment thereof, blocks the generation or activity of the C5a and/or C5b active fragments of a C5 protein (e.g., a human C5 protein). Through this blocking effect, the antibodies inhibit, e.g., the pro-inflammatory effects of C5a and the generation of the C5b-9 membrane attack complex (MAC) at the surface of a cell.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2^{nd} Edition,” 135-240, Springfield, Ill., C C Thomas (1961), pages 135-139, or a conventional variation of that assay such as the chicken erythrocyte hemolysis method as described in, e.g., Hillmen et al. (2004) N Engl J Med 350(6):552. Methods for determining whether a candidate compound inhibits the cleavage of human C5 into forms C5a and C5b are known in the art and described in Evans et al. (1995) Mol Immunol 32(16):1183-95. For example, the concentration and/or physiologic activity of C5a and C5b in a body fluid can be measured by methods well known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of an anti-C5 antibody, or antigen binding fragment thereof, on complement activation. In order to determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-05b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microas say wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55, or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90, or 95) % of the complement inhibitory activity of eculizumab in a hemolytic assay or CH50eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400, 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

### III. Compositions

Also, provided herein are compositions (e.g., formulations) comprising an anti-C5 antibody, or antigen binding fragment thereof (e.g., ravulizumab), and a hyaluronidase (e.g., a recombinant human hyaluronidase, such as rHuPH20) for use in the methods described herein.

In one embodiment, the hyaluronidase (e.g., a recombinant human hyaluronidase) and antibody, or antigen-binding fragment thereof, are combined in a single formulation. For example, the hyaluronidase (e.g., a recombinant human hyaluronidase) and antibody, or antigen-binding fragment thereof, are mixed and co-administered in a single formulation.

The compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

In one embodiment, the hyaluronidase is rHuPH20 (ENHANZE®) at a concentration of 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000, 30,500, 31,000, 31,500, 32,000, 32,500, 33,000, 33,500, 34,000, 34,500, 35,000, 35,500, 36,000, 36,500, 37,000, 37,500, 38,000, 38, 500, 39,000, 39,500, 40,000, 40,500, 45,000, or 50,000 units. In a particular embodiment, rHuPH20 is at a concentration of 10,000 units. In another particular embodiment, rHuPH20 is at a concentration of 20,000 units. In another particular embodiment, rHuPH20 is at a concentration of 40,000 units.

In another embodiment, the antibody, or antigen-binding fragment thereof, is ravulizumab at a dose of 500 mg, 600 mg, 700 mg, 800 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, 5,000 mg, 5,100 mg, 5,200 mg, 5,300 mg, 5,400 mg, 5,500 mg, 5,600 mg, 5,700 mg, 5,800 mg, 5,900 mg, 6,000 mg, or 7,000 mg. In a particular embodiment, the antibody, or antigen-binding fragment thereof, is ravulizumab at a dose of 500 mg. In another particular embodiment, the antibody, or antigen-binding fragment thereof, is ravulizumab at a dose of 1000 mg. In another particular embodiment, the antibody, or antigen-binding fragment thereof, is ravulizumab at a dose of 2000 mg.

In another embodiment, the composition is a single formulation comprising ravulizumab at 500 mg and 10,000 units of rHuPH20. In another embodiment, the composition is a single formulation comprising ravulizumab at 1000 mg and 20,000 units of rHuPH20. In another embodiment, the composition is a single formulation comprising ravulizumab at 2000 mg and 40,000 units of rHuPH20.

In another embodiment, the composition comprises (a) a formulation comprising 1100 mg of ravulizumab, 50 mM sodium phosphate, 25 mM arginine, 5% sucrose, and 0.05% polysorbate 80) mixed with (b) a formulation comprising 110 kU/mL of rHuPH20, 130 mM sodium chloride, 10 mM L-Histidine/hydrochloride, 10 mM L-Methionine and 0.2% w/w polysorbate 80.

### IV. Methods

Provided herein are methods for treating a human patient with a complement-associated condition (e.g., PNH or aHUS) by subcutaneously administering (e.g., co-administering) to the patient a hyaluronidase (e.g., a recombinant human hyaluronidase) and an anti-C5 antibody, or antigen binding fragment thereof. Co-administration of a hyaluronidase (e.g., a recombinant human hyaluronidase) and an anti-C5 antibody, or antigen binding fragment thereof, facilitates a larger amount of the anti-C5 antibody, or antigen binding fragment thereof, to be administered in a single time, thereby allowing less frequent dosing. In addition, this particular combination is particularly advantageous in that it provides patients with a self-administered dosing option that eliminates the patient burden associated with intravenous (IV) infusions (e.g., loss of work time, disruption of routine associated with dosing frequency, and prolonged infusion times).

As used herein, the term “subject” or “patient” is a human patient (e.g., a patient having complement-associated condition, such as PNH or aHUS).

As used herein, co-administration (also known as adjunctive or combined administration) includes simultaneous administration of the compounds in the same or different dosage form, or separate administration of the compounds (e.g., sequential administration). In one embodiment, the hyaluronidase (e.g., a recombinant human hyaluronidase) and antibody, or antigen-binding fragment thereof, are administered simultaneously in separate formulations. In another embodiment, the hyaluronidase (e.g., a recombinant human hyaluronidase) and anti-C5 antibody, or antigen-binding fragment thereof, are administered sequentially (e.g., as separate formulations). For example, the hyaluronidase (e.g., a recombinant human hyaluronidase) can be administered first followed by (e.g., immediately followed by) the administration of the anti-C5 antibody, or antigen-binding fragment thereof, or vice versa. Such concurrent or sequential administration preferably results in both the hyaluronidase (e.g., a recombinant human hyaluronidase) and anti-C5 antibody, or antigen-binding fragment thereof, being simultaneously present in treated patients.

In another embodiment, the hyaluronidase (e.g., a recombinant human hyaluronidase) and antibody, or antigen-binding fragment thereof, are administered simultaneously in a single formulation. For example, the hyaluronidase (e.g., a recombinant human hyaluronidase) and antibody, or antigen-binding fragment thereof, can be mixed and co-administered in a single formulation.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. In the context of PNH, for example, effective treatment may refer to alleviation of one more symptoms selected from the group consisting of fatigue, abdominal pain, dyspnea, dysphagia, chest pain, and/or erectile dysfunction. In the context of aHUS, for example, effective treatment may refer to the alleviation of one or more symptoms selected from the group consisting of severe hypertension, proteinuria, uremia, lethargy/fatigue, irritability, thrombocytopenia, microangiopathic hemolytic anemia, and/or renal function impairment (e.g., acute renal failure).

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In one example, an “effective amount” is the amount of anti-C5 antibody, or antigen binding fragment thereof, clinically proven to alleviate at least one symptom of PNH (e.g., fatigue, abdominal pain, dyspnea, dysphagia, chest pain, or erectile dysfunction) or at least one symptom of aHUS (e.g., severe hypertension, proteinuria, uremia, lethargy/fatigue, irritability, thrombocytopenia, microangiopathic hemolytic anemia, and renal function impairment (e.g., acute renal failure)). An effective amount can be administered in one or more administrations.

In one embodiment, the recombinant human hyaluronidase is rHuPH20 (ENHANZE®) administered at a concentration of 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000, 30,500, 31,000, 31,500, 32,000, 32,500, 33,000, 33,500, 34,000, 34,500, 35,000, 35,500, 36,000, 36,500, 37,000, 37,500, 38,000, 38, 500, 39,000, 39,500, 40,000, 40,500, 45,000, or 50,000 units. In a particular embodiment, rHuPH20 is administered at a concentration of 10,000 units. In another particular embodiment, rHuPH20 is administered at a concentration of 20,000 units. In another particular embodiment, rHuPH20 is administered at a concentration of 40,000 units.

In another embodiment, the antibody, or antigen-binding fragment thereof, is ravulizumab administered at a dose of 500 mg, 600 mg, 700 mg, 800 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, 5,000 mg, 5,100 mg, 5,200 mg, 5,300 mg, 5,400 mg, 5,500 mg, 5,600 mg, 5,700 mg, 5,800 mg, 5,900 mg, 6,000 mg, or 7,000 mg. In a particular embodiment, the antibody, or antigen-binding fragment thereof, is ravulizumab administered at a dose of 500 mg. In another particular embodiment, the antibody, or antigen-binding fragment thereof, is ravulizumab administered at a dose of 1000 mg. In another particular embodiment, the antibody, or antigen-binding fragment thereof, is ravulizumab administered at a dose of 2000 mg.

In another embodiment, the antibody, or antigen-binding fragment thereof, is ravulizumab and the hyaluronidase (e.g., a recombinant human hyaluronidase) is rHuPH20, and the patient is separately administered ravulizumab at 500 mg and 10,000 units of rHuPH20 (e.g., sequentially or simultaneously as separate formulations). In one embodiment, rHuPH20 is administered just prior to administration of ravulizumab. In another embodiment, ravulizumab is administered just prior to administration of rHuPH20.

In another embodiment, the antibody, or antigen-binding fragment thereof, is ravulizumab and the hyaluronidase (e.g., a recombinant human hyaluronidase) is rHuPH20, and the patient is administered a single formulation comprising ravulizumab at 500 mg and 10,000 units of rHuPH20.

In another embodiment, the antibody, or antigen-binding fragment thereof, is ravulizumab and the recombinant human hyaluronidase is rHuPH20, and the patient is separately administered ravulizumab at 1000 mg and 20,000 units of rHuPH20 (e.g., sequentially or simultaneously as separate formulations). In one embodiment, rHuPH20 is administered just prior to administration of ravulizumab. In another embodiment, ravulizumab is administered just prior to administration of rHuPH20.

In another embodiment, the antibody, or antigen-binding fragment thereof, is ravulizumab and the recombinant human hyaluronidase is rHuPH20, and the patient is administered a single formulation comprising ravulizumab at 1000 mg and 20,000 units of rHuPH20.

In another embodiment, the antibody, or antigen-binding fragment thereof, is ravulizumab and the recombinant human hyaluronidase is rHuPH20, and the patient is separately administered ravulizumab at 2000 mg and 40,000 units of rHuPH20 (e.g., sequentially or simultaneously as separate formulations). In one embodiment, rHuPH20 is administered just prior to administration of ravulizumab. In another embodiment, ravulizumab is administered just prior to administration of rHuPH20.

In another embodiment, the antibody, or antigen-binding fragment thereof, is ravulizumab and the recombinant human hyaluronidase is rHuPH20, and the patient is administered a single formulation comprising ravulizumab at 2000 mg and 40,000 units of rHuPH20.

In another embodiment, the hyaluronidase (e.g., a recombinant human hyaluronidase) and anti-C5 antibody, or antigen binding fragment thereof, are administered to the patient once every two weeks, once every three weeks, once a month, once every month and a half, once every two months, or once every three months.

Subcutaneous administration of a hyaluronidase (e.g., rHuPH20) and an anti-C5 antibody, or antigen binding fragment thereof, (e.g., ravulizumab) according to the methods described herein can be accomplished by any suitable means. In addition, the hyaluronidase (e.g., a recombinant human hyaluronidase) and anti-C5 antibody, or antigen binding fragment thereof, can be administered subcutaneously by a medical professional or self-administered. In one embodiment, the hyaluronidase (e.g., a recombinant human hyaluronidase) and antibody, or antigen-binding fragment thereof, are subcutaneously administered to the patient via an infusion pump. In another embodiment, the hyaluronidase (e.g., a recombinant human hyaluronidase) and anti-C5 antibody, or antigen binding fragment thereof, is administered subcutaneously using an on-body delivery system (OBDS).

In some embodiments, the patients treated according to the methods described herein have been vaccinated against meningococcal infections prior to initiating treatment. In one embodiment, patients treated according to the methods described herein are vaccinated against meningococcal serotypes A, C, Y, W135, and/or B. In another embodiment, patients treated according to the methods described herein receive the MCV4 vaccination at least 56 days prior to dosing with the anti-C5 antibody, or antigen binding fragment thereof (e.g., ravulizumab). In one embodiment, where a patient has not already been vaccinated for serotype B meningococcal infections, the patient is vaccinated for serotype B meningococcal infections at least 56 days prior to initiating treatment, with a booster administered at least 28 days prior to initiating treatment.

In some embodiments, the patient is administered one or more additional therapeutic agents prior to and/or during treatment. For example, in one embodiment, the patient is administered an antibiotic (e.g., 500 mg of penicillin orally twice daily or ciprofloxacin) prior to and/or during treatment. In another embodiment, the hyaluronidase (e.g., a recombinant human hyaluronidase) and anti-C5 antibody, or antigen binding fragment thereof, are administered in combination with no more than three additional agents. In another embodiment, the hyaluronidase (e.g., a recombinant human hyaluronidase) and anti-C5 antibody, or antigen binding fragment thereof, are administered in combination with no more than two additional agents. In another embodiment, the hyaluronidase (e.g., a recombinant human hyaluronidase) and anti-C5 antibody, or antigen binding fragment thereof, are administered in combination with no more than one additional agent. In another embodiment, no additional agents are administered in combination with the hyaluronidase (e.g., a recombinant human hyaluronidase) and anti-C5 antibody, or antigen binding fragment thereof.

In another aspect, the treatment regimens described herein are sufficient to maintain particular serum trough concentrations of the anti-C5 antibody, or antigen binding fragment thereof. For example, in one embodiment, the treatment maintains a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, or 400 μg/ml or greater. In another embodiment, the treatment maintains a serum trough concentration of the anti-C5 antibody, or antigen binding fragment thereof, of 100 μg/ml or greater. In another embodiment, the treatment maintains a serum trough concentration of the anti-C5 antibody, or antigen binding fragment thereof, of 150 μg/ml or greater. In another embodiment, the treatment maintains a serum trough concentration of the anti-C5 antibody, or antigen binding fragment thereof, of 200 μg/ml or greater. In another embodiment, the treatment maintains a serum trough concentration of the anti-C5 antibody, or antigen binding fragment thereof, of 250 μg/ml or greater. In another embodiment, the treatment maintains a serum trough concentration of the anti-C5 antibody, or antigen binding fragment thereof, of 300 μg/ml or greater. In another embodiment, the treatment maintains a serum trough concentration of the anti-C5 antibody, or antigen binding fragment thereof, of between 100 μg/ml and 200 μg/ml. In another embodiment, the treatment maintains a serum trough concentration of the anti-C5 antibody, or antigen binding fragment thereof, of about 175 μg/ml.

In some embodiments, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

### V. Complement-Associated Conditions

Exemplary complement-associated conditions that can be treated according to the methods described herein include, but are not limited to, rheumatoid arthritis, antiphospholipid antibody syndrome, lupus nephritis, ischemia-reperfusion injury, atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome.

In one embodiment, the complement-associated condition is paroxysmal nocturnal hemoglobinuria (PNH). PNH is an acquired hemolytic disorder that occurs most frequently in adults (Brodsky R A., Blood. 2015; 126:2459-65). The disease begins with the clonal expansion of a hematopoietic stem cell that has acquired a somatic mutation in the PIGA gene (Brodsky R A., Blood. 2014; 124:2804-1). Consequently, PNH blood cells lack the glycophosphatidylinositol (GPI) anchor protein and are deficient in the membrane-bound complement inhibitory proteins CD55 and CD59. In the absence of CD55, there is increased deposition of complement protein C3 cleavage products on blood cell membrane surfaces, in turn leading to cleavage of C5 into C5a and C5b. The pathology and clinical presentations in patients with PNH are driven by uncontrolled terminal complement activation.

C5a is a potent anaphylatoxin, chemotactic factor, and cell-activating molecule that mediates multiple pro-inflammatory and pro-thrombotic activities (Matis L A, et al., Nat. Med. 1995; 1:839-42; Prodinger et al., Complement. In: Paul W E, editor. Fundamental immunology (4th ed). Philadelphia: Lippincott-Raven Publishers; 1999. p. 967-95). C5b recruits the terminal complement components C6, C7, C8, and C9 to form the pro-inflammatory, pro-thrombotic cytolytic pore molecule C5b-9, a process that under normal circumstances would be blocked on the red blood cell (RBC) membrane by CD59. In patients with PNH, however, these final steps proceed unchecked, culminating in hemolysis and the release of free hemoglobin, as well as platelet activation (Hill, et al., Blood 2013; 121:4985-96). The signs and symptoms of PNH can be attributed to chronic, uncontrolled complement C5 cleavage, and release of C5a and C5b-9 leading to RBC hemolysis, which together result in (Hill, et al., Blood 2013; 121:4985-96; Brodsky R A., Blood. 2014; 124:2804-1): release of intracellular free hemoglobin and lactate dehydrogenase (LDH) into circulation as a direct consequence of hemolysis, irreversible binding to and inactivation of nitric oxide (NO) by hemoglobin, and inhibition of NO synthesis, vasoconstriction and tissue-bed ischemia due to absence of vasodilatory NO, as well as possible microthrombi manifesting as abdominal pain, dysphagia, and erectile dysfunction, platelet activation, and/or pro-inflammatory and prothrombotic state. A substantial proportion of patients with PNH experience renal dysfunction and pulmonary hypertension (Hillmen, et al., Am J Hematol. 2010; 85:553-9. [erratum in Am J Hematol. 2010; 85:911.]; Hill, et al., Br. J Haematol. 2012; 158:409-14; Hill, et al., Blood 2013; 121:4985-96). Patients also experience venous or arterial thrombosis in diverse sites, including the abdomen or central nervous system (Brodsky R A., Blood. 2014; 124:2804-1).

In another embodiment, the complement-associated condition is atypical hemolytic uremic syndrome (aHUS). The pathology and clinical presentations of patients with aHUS are also driven by terminal complement activation. More specifically, activation of C5 and dysregulation of complement activation lead to endothelial damage, platelet consumption, and thrombotic microangiopathic (TMA) events, characterized by thrombocytopenia, mechanical intravascular hemolysis, and kidney injury. Importantly, approximately 20% of patients experience extra-renal manifestations of disease as well, including central nervous system, cardiac, gastrointestinal, distal extremities, and severe systemic organ involvement (Loirat, et al., Orphanet. J. Rare Dis. 2011; 6:60). Symptoms of aHUS are well-known to those of skill in the art of rare disease or kidney disease medicine and include, e.g., severe hypertension, proteinuria, uremia, lethargy/fatigue, irritability, thrombocytopenia, microangiopathic hemolytic anemia, and renal function impairment (e.g., acute renal failure).

aHUS can be genetic, acquired, or idiopathic. aHUS can be considered genetic when two or more (e.g., three, four, five, or six or more) members of the same family are affected by the disease at least six months apart and exposure to a common triggering agent has been excluded, or when one or more aHUS-associated gene mutations (e.g., one or more mutations in CFH, MCP/CD46, CFB, or CFI) are identified in a subject. For example, a subject can have CFH-associated aHUS, CFB-associated aHUS, CFI-associated aHUS, or MCP-associated aHUS. Up to 30% of genetic aHUS is associated with mutations in CFH, 12% with mutations in MCP, 5-10% with mutations in CFI, and less than 2% with mutations in CFB. Genetic aHUS can be multiplex (i.e., familial; two or more affected family members) or simplex (i.e., a single occurrence in a family). aHUS can be considered acquired when an underlying environmental factor (e.g., a drug, systemic disease, or viral or bacterial agents that do not result in Shiga-like exotoxins) or trigger can be identified. aHUS can be considered idiopathic when no trigger (genetic or environmental) is evident.

Laboratory tests can be performed to determine whether a human subject has thrombocytopenia, microangiopathic hemolytic anemia, or acute renal insufficiency. Thrombocytopenia can be diagnosed by a medical professional as one or more of: (i) a platelet count that is less than 150,000/mm³ (e.g., less than 60,000/mm³); (ii) a reduction in platelet survival time that is reduced, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than 10 mg/dL (e.g., less than 6.5 mg/dL); (ii) increased serum lactate dehydrogenase (LDH) concentrations (>460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668). Blood concentrations of C3 and C4 can also be used as a measure of complement activation or dysregulation. In addition, a subject's condition can be further characterized by identifying the subject as harboring one or more mutations in a gene associated with aHUS such as CFI, CFB, CFH, or MCP (supra). Suitable methods for detecting a mutation in a gene include, e.g., DNA sequencing and nucleic acid array techniques. See, e.g., Breslin et al. (2006) Clin Am Soc Nephrol 1:88-99 and Goicoechea de Jorge et al. (2007) Proc Natl Acad Sci USA 104:240-245.

### VI. Outcomes

The efficacy of the treatment methods provided herein can be assessed using any suitable means. In one embodiment, the treatment results in terminal complement inhibition. In another embodiment, the methods described herein result in the amelioration of at least one symptom of the complement-associated disease. For example, in the context of PNH, the treatment may alleviate of one more symptoms selected from the group consisting of fatigue, abdominal pain, dyspnea, dysphagia, chest pain, and/or erectile dysfunction). In the context of aHUS, for example, the treatment may alleviate one or more symptoms selected from the group consisting of severe hypertension, proteinuria, uremia, lethargy/fatigue, irritability, thrombocytopenia, microangiopathic hemolytic anemia, and/or renal function impairment (e.g., acute renal failure).

In another embodiment, lactate dehydrogenase (LDH) levels are used to evaluate responsiveness to a therapy (e.g., a reduction of hemolysis as assessed by lactate dehydrogenase (LDH) levels is indicative of an improvement in at least one sign of PNH). For example, in one embodiment, the treatments described herein result in a normalization of LDH levels.

LDH is a marker of intravascular hemolysis (Hill, A. et al., Br. J. Haematol., 149:414-25, 2010; Hillmen, P. et al., N. Engl. J. Med., 350:552-9, 2004; Parker, C. et al., Blood, 106:3699-709, 2005). Red blood cells contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):0:359 Abs.0903, 2008; Kanakura, Y. et al., Int. J. Hematol., 93:36-46, 2011). LDH concentration obtained at baseline and then serially throughout a treatment period, is an important measure of hemolysis. Baseline levels of cell-free plasma hemoglobin are highly elevated in patients with PNH with LDH≥1.5-fold above the upper limit of normal (LDH≥1.5×ULN), with a significant correlation between LDH and cell-free plasma hemoglobin (Hillmen, P. et al., N. Engl. J. Med., 355:1233-43, 2006). The normal LDH value range is 105-333 IU/L (international units per liter).

Published data support LDH as a reliable, objective, and direct measure of intravascular hemolysis in patients with PNH and is considered by experts to be the best measure of complement-mediated hemolysis, the hallmark of PNH disease activity (Dale J. et al., Acta Med Scand., 191(1-2):133-136, 1972; Parker C. et al., Blood. 106(12):3699-3709, 2005; Canalejo K et al., Int J Lab Hemat., 36(2):1213-1221, 2013). Results from the eculizumab clinical program showed that LDH concentrations remained markedly elevated and unchanged in untreated (placebo) patients, while eculizumab-treated patients had an immediate reduction (within 1 week following initiation of treatment) in serum LDH to normal or near normal levels (Brodsky R A et al., Blood, 111(4):1840-1847, 2008; Hillmen P et al., Am J Hematol., 85(8):553-559, 2010. Erratum in Am J Hematol. 2010; 85(11):911). This reduction mirrored a rapid reduction in symptoms and improvement in fatigue (Hillmen P et al., Am J Hematol., 85(8):553-559, 2010; Brodsky R A et al., Blood, 111(4):1840-1847, 2008).

LDH levels can be measured using any suitable test or assay, such as those described by Ferri F F, ed. Ferri's Clinical Advisor 2014. Philadelphia: Pa: Elsevier Mosby; 2014: Section IV—Laboratory tests and interpretation of results. LDH concentration can be measured in various samples obtained from a patient, in particular, serum samples. As used herein, the term “sample” refers to biological material from a subject. Although serum LDH concentration is of interest, samples can be derived from other sources, including, for example, single cells, multiple cells, tissues, tumors, biological fluids, biological molecules or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are known in the art and can be readily adapted to obtain a sample that is compatible with the method utilized.

In one embodiment, lactate dehydrogenase (LDH) levels are used to evaluate responsiveness to a therapy (e.g., a reduction of hemolysis as assessed by lactate dehydrogenase (LDH) levels is indicative of an improvement in at least one sign of PNH). For example, in one embodiment, the treatments described herein result in a normalization of LDH levels. In one embodiment, patients treated according to the disclosed methods experience reductions in LDH levels to within normal levels or to within 10%, 20%, 30%, 40% or within 50% below what is considered the upper limit of normal level (e.g., within 105-333 IU/L (international units per liter). In another embodiment, the patient's LDH levels are ≥1.5 fold above the upper limit of normal (LDH≥1.5×ULN) prior to initiating treatment. In another embodiment, the patient's LDH levels are normalized throughout maintenance period of treatment. In another embodiment, the treated patient's LDH levels are normalized at least at least 95% of the time while on the maintenance period of treatment. In another embodiment, the treated patient's LDH levels are normalized at least at least 90%, 85% or 80% of the time while on the maintenance period of treatment. In one embodiment, the patient's LDH levels are ≥1.5 fold above the upper limit of normal (LDH≥1.5×ULN) prior to initiating treatment.

In another embodiment, the treatment produces a reduction in the need for blood transfusions. In another embodiment, the treatment produces an increase in transfusion avoidance. In another embodiment, the treatment produces an increase of at least 50% in transfusion avoidance. In another embodiment, the treatment produces an increase of at least 60% in transfusion avoidance. In another embodiment, the treatment produces a greater than 70% increase in transfusion avoidance. In all cases the transfusion avoidance is measured against pretreatment frequency for the requirement to receive transfusions.

In another embodiment, the treatment produces a reduction in major adverse vascular events (MAVEs) (e.g., thrombophlebitis/deep vein thrombosis, pulmonary embolus, myocardial infarction, transient ischemic attack, unstable angina, renal vein thrombosis/renal artery thrombosis/glomerular thrombosis, renal infarction, acute peripheral vascular occlusion, mesenteric/visceral vein/arterial thrombosis or infarction, hepatic/portal vein thrombosis, cerebral arterial occlusion/cerebrovascular accident, cerebral venous occlusion, renal arterial thrombosis, or multi-infarct dementia), as described in further detail in the Examples.

In another embodiment, the treatment produces a shift toward normal levels of a hemolysis-related hematologic biomarker selected from the group consisting of free hemoglobin, haptoglobin, reticulocyte count, PNH red blood cell (RBC) clone and D-dimer. In another embodiment, the treatment produces an increase in hemoglobin stabilization from the patient's pre-treatment baseline.

In another embodiment, the treatment produces a shift toward normal levels of a chronic disease associated biomarker selected from the group consisting estimated glomerular filtration rate (eGFR) and spot urine:albumin:creatinine and plasma brain natriuretic peptide (BNP).

In another embodiment, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale, and described in further detail in the Examples. In another embodiment, the treatment produces a change from baseline in quality of life, as assessed via the Functional Assessment of Chronic Illness Therapy (FACIT)-Fatigue Scale, version 4 and the European Organisation for Research and Treatment of Cancer, Quality of Life Questionnaire-Core 30 Scale by at least 7 points from the patients untreated baseline score.

### VII. Kits

Also provided are kits that include an anti-C5 antibody, or antigen binding fragment thereof (e.g., ravulizumab), and a hyaluronidase (e.g., rHuPH20), in therapeutically effective amounts adapted for use in the methods described herein. For example, in one embodiment, a kit for treating a complement-associated condition in a human patient is provided, the kit comprising: (a) a dose of an anti-C5 antibody, or antigen binding fragment thereof, (b) a dose of a hyaluronidase (e.g., a recombinant human hyaluronidase), and (c) instructions for using the anti-C5 antibody, or antigen binding fragment thereof, and hyaluronidase (e.g., a recombinant human hyaluronidase) in the method of any one of the preceding claims. In one embodiment, the anti-C5 antibody is ravulizumab. In another embodiment, the hyaluronidase is rHuPH20 (ENHANZE®). The kits optionally also can include instructions, e.g., comprising administration schedules, to allow a practitioner (e.g., a physician, nurse, or patient) to administer the composition contained therein to administer the composition to a patient having a complement-associated condition, such as PNH or aHUS. The kit also can include a syringe or an on-body delivery system (OBDS).

### VIII. Devices

Further provided is a device comprising a prefilled cartridge of an anti-C5 antibody, or antigen binding fragment thereof (e.g., ravulizumab), and a hyaluronidase (e.g., rHuPH20) for subcutaneous administration co-packaged with an on-body injector. In one embodiment, the device is sterile, for single use, disposable, and/or electro-mechanical.

An exemplary device for use in conjunction with ravulizumab for subcutaneous administration as described herein is the on-body delivery system (OBDS) manufactured by West Pharmaceuticals, Inc., which is currently approved for use with evolocumab (Repatha®) as a combination agent in the United States and CE marked in the European Union as a class IIA Medical Device. The device is a compact, sterile, single-use, disposable, electro-mechanical (battery powered, microprocessor controlled), investigational medical device with a 29-gauge integrated needle (manufactured by West Pharmaceuticals, Inc.) designed to be used together with a prefilled stoppered Crystal Zenith® cartridge with a piston and telescopic screw assembly (TSA).

The following examples are merely illustrative and should not be construed as limiting the scope of this disclosure in any way as many variations and equivalents will become apparent to those skilled in the art upon reading the present disclosure.

The contents of all references, Genbank entries, patents and published patent applications cited throughout this application are expressly incorporated herein by reference.

### EXAMPLES

### Example 1: Clinical Trial

A partially randomized, sequential cohort, single ascending dose study is conducted in healthy adult volunteers to assess the safety, tolerability, pharmacokinetics, pharmacodynamics, and immunogenicity of subcutaneous ravulizumab coadministered with rHuPH20.

1. Objectives

The primary objective is to estimate the absolute bioavailability of ravulizumab subcutaneous (SC)/recombinant human hyaluronidase PH20 (rHuPH20) (also referred to herein as “ravulizumab SC/rHuPH20”), as well as to assess its safety and tolerability. Endpoints include the use of ravulizumab serum concentration to determine the geometric mean ratio (GMR) of the area under the concentration time curve (AUC) values. Safety is assessed by incidence of treatment-emergent adverse events (TEAEs) and serious adverse events (SAEs), physical examination, vital sign measurements, clinical laboratory and electrocardiogram results, and measurement of antidrug antibodies (ADA).

Secondary objectives include estimating the relative bioavailability of ravulizumab SC/rHuPH20 compared with ravulizumab SC. The serum concentration of ravulizumab is used to determine the GMR of the AUC values. Another secondary objective is to explore the pharmacodynamics (PD) effects of ravulizumab SC co-administered with rHuPH20. This includes assessing the change in serum levels of total and free C5 concentrations over time and the change in ex vivo chicken red blood cell (cRBC) hemolysis activity over time.

2. Overall Design

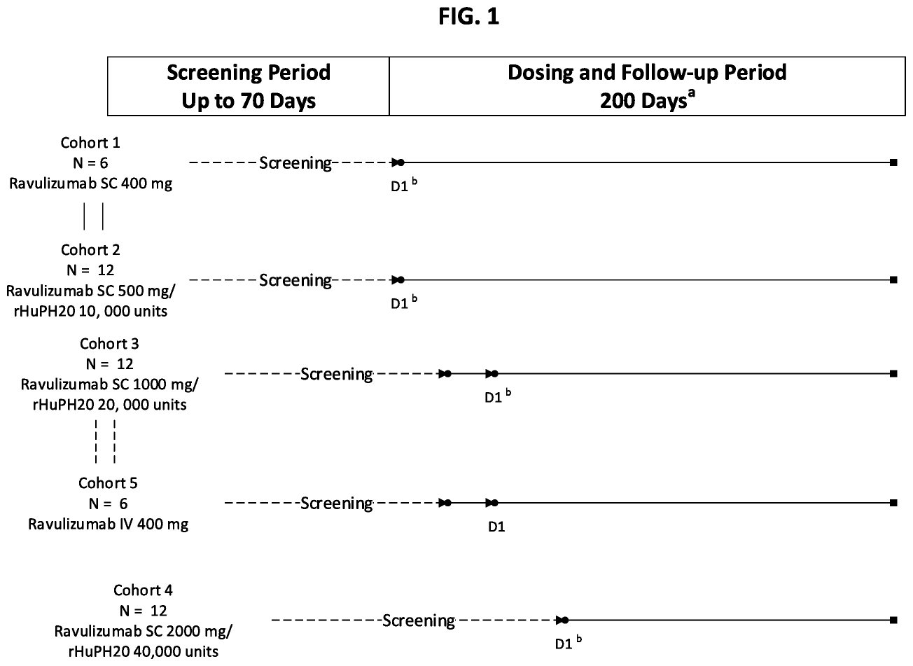

This is a Phase 1 study designed to evaluate the safety, tolerability, pharmacokinetics (PK), pharmacodynamics (PD), immunogenicity, and absolute and relative bioavailability of single ascending doses of ravulizumab SC coadministered with recombinant human hyaluronidase PH20 (rHuPH20) compared to a single dose of ravulizumab IV 400 mg or a single dose of ravulizumab SC 400 mg in 48 healthy adult subjects. A schematic depicting the overall study design is set forth in FIG. 1. Five treatment cohorts are included. The doses for each cohort are set forth in Table 1. A Safety Review Committee (SRC) evaluates the study data for subject safety and makes recommendations on dose escalation, dose modification, or termination of the study. When cohorts are enrolled in parallel, subject assignment to a cohort is done through randomization. Subjects are randomly assigned up to 7 days prior to dosing on Day 1. The study is conducted at a single site in the United Kingdom.

TABLE 1
Study Design

| | # of | | | Planned |
|---|---|---|---|---|
| Cohort | Subjects | Study Drug | Route (SC/IV) | Dose |
| 1 | 6 | ravulizumab | Subcutaneous (SC) | 400 mg |
| 2 | 12 | ravulizumab/ | Intravenous (IV) | 500 mg/ |
| | | rHuPH20 | | 10,000 units |
| 3 | 12 | ravulizumab/ | Subcutaneous (SC) | 1000 mg/ |
| | | rHuPH20 | | 20,000 units |
| 4 | 12 | ravulizumab/ | Subcutaneous (SC) | 2000 mg/ |
| | | rHuPH20 | | 40,000 units |
| 5 | 6 | ravulizumab | Intravenous (IV) | 400mg |

Eighteen subjects are randomly assigned in a 1:2 ratio between the first 2 cohorts to receive either a single dose of ravulizumab SC 400 mg (Cohort 1, n=6) or a single dose of ravulizumab SC 500 mg/rHuPH20 10,000 units (Cohort 2, n=12). The SRC reviews Cohort 2 ravulizumab SC/rHuPH20 safety data and makes a recommendation for escalating the dose of the ravulizumab SC/rHuPH20 combination cohort.

If the SRC recommendation following the review of data from Cohort 2 is to initiate the next cohort and proceed with the planned dose escalation, 18 subjects are randomly assigned in a 2:1 ratio between Cohort 3 (n=12) and Cohort 5 (n=6) to receive either a single dose of ravulizumab SC 1000 mg/rHuPH20 20,000 units or a single dose of ravulizumab IV 400 mg, respectively. The SRC subsequently reviews Cohort 3 ravulizumab SC/rHuPH20 safety data and makes a recommendation to proceed with planned dose escalation in Cohort 4 or a reduced dose in Cohort 4. Whether the recommendation is to initiate Cohort 4 with the planned dose escalation or at a reduced dose, 12 subjects are enrolled in Cohort 4 to receive a single dose of ravulizumab SC/rHuPH20 (dose to be determined based on SRC recommendation).

If the SRC recommendation following the review of ravulizumab SC/rHuPH20 safety data from Cohort 2 is to proceed with Cohort 3 at a reduced dose, 18 subjects are randomly assigned in a 2:1 ratio between Cohort 3 (n=12) and Cohort 5 (n=6) to receive either a single dose of ravulizumab SC/rHuPH20 (dose to be determined based on SRC recommendation) or a single dose of ravulizumab IV 400 mg, respectively. In this scenario, Cohort 4, if conducted, is enrolled at a reduced dose, following completion of Cohort 3 based on SRC review of ravulizumab SC/rHuPH20 safety data from Cohort 3 and a favorable recommendation to enroll subjects in Cohort 4. This protocol allows for reduced doses to be administered to subjects in Cohorts 3 and 4, based on SRC recommendation, without a protocol amendment. If a reduced dose is administered, the SRC reduced dose recommendation is documented on the escalation/progression approval form.

If the SRC determines that no further ravulizumab SC/rHuPH20 combination dosing cohorts should be enrolled following Cohort 2, then Cohort 5 may still be enrolled as a stand-alone cohort at the discretion of the Sponsor.

Sentinel dosing is employed in Cohorts 1 through 4 (i.e., 2 subjects in a cohort with 12 subjects and 1 subject in a cohort with 6 subjects are dosed prior to dosing the remaining subjects within the cohort). The remaining subjects in a cohort are dosed at least 24 hours following dosing of the sentinel subjects.

The SRC reviews all available safety data through 168 hour (Day 8) assessments to determine initiation of the next dose cohort.

At the Sponsor's discretion, and after consultation with the SRC, up to 16 additional subjects may be enrolled as replacement subjects if a subject discontinues prior to Day 50 for reasons other than drug-related adverse events.

The planned study duration is approximately 39 weeks: up to 70 days for screening and approximately 200 days for dosing and follow-up. For the first 5 days during the Dosing and Follow-up Period, subjects are admitted to an inpatient facility. Dosing is staggered within and between cohorts, but the end of study for each individual subject is anticipated to be Day 200 or the time point at which complement activity has normalized, if later than Day 200.

The SRC reviews all available safety data for at least the first 168 hours after dose administration from a given cohort to determine whether to initiate the next cohort and escalate the dose of ravulizumab SC/rHuPH20. Data through 168 hours must be available for at least 11 of the 12 subjects. Dose escalation occurs based on the recommendation of the SRC and only applies to Cohorts 3 and 4. SRC decisions are documented in study minutes and archived in the trial master file.

3. End of Study Definition

A subject is considered to have completed the study if he/she has completed all visits of the study including the last scheduled visit specified in the Schedule of Activities set forth in Table 2 and Table 3. The end of the study is defined as the last scheduled visit for the last subject, specified in the Schedules of Activities. No further study assessments beyond CH50 evaluation, as needed based on individual subject results, are performed after Day 200.

TABLE 2
Schedule of Activities - Screening Through Visit 1

| | | | | Visit 1 | | | | | | | | |
|---|---|---|---|---|---|---|---|---|---|---|---|---|
| | | | | Day 1 | | | | | | | | |
| | Screening | Day −1 | | | | 30 min | 2 h | 4 h | 8 h | | | |
| Study Day | Day −70 | Admit | | 0 h | EOI | post | post | post | post | Day 2 | Day 3 | Day 5 |
| Assessments^{a} | to Day −2 | Day −1 | Predose | (SOI) | (SC)^{b} | SOI | SOI | SOI | SOI | 24 h | 48 h | 96 h |
| Status (OP or CRU) | OP | Admit | CRU | CRU | CRU | CRU | CRU | CRU | CRU | CRU | CRU | CRU^{c} |
| Informed consent^{d} | X | | | | | | | | | | | |
| MCV4 | X | | | | | | | | | | | |
| immunization^{e} | | | | | | | | | | | | |
| Meningococcal | X | | | | | | | | | | | |
| serogroup B | | | | | | | | | | | | |
| immunization^{e} | | | | | | | | | | | | |
| Vaccine titer | X | | | | | | | | | | | |
| (meningococcal | | | | | | | | | | | | |
| serogroups A, C, | | | | | | | | | | | | |
| W135, and Y)^{f} | | | | | | | | | | | | |
| Medical history | X | | | | | | | | | | | |
| and demographics | | | | | | | | | | | | |
| Physical | X | X | | | | | | | | | | X |
| examination | | | | | | | | | | | | |
| Height, weight, | X | | | | | | | | | | | |
| and BMI | | | | | | | | | | | | |
| QuantiFERON ®- | X | | | | | | | | | | | |
| TB test | | | | | | | | | | | | |
| Biochemistry | X | X | | | | | | | | X | | |
| Hematology | X | X | | | | | | | | X | | |
| Coagulation | X | X | | | | | | | | X | | |
| Hepatitis B and C | X | | | | | | | | | | | |
| screen | | | | | | | | | | | | |
| HIV (types 1 and 2) | X | | | | | | | | | | | |
| screen | | | | | | | | | | | | |
| Complement | X | | | | | | | | | | | |
| activity^{g} | | | | | | | | | | | | |
| CH50 | | | X | | | | | | | | | |
| Serum pregnancy | X | X | | | | | | | | | | |
| test^{h} | | | | | | | | | | | | |
| Alcohol breath test | X | X | | | | | | | | | | |
| Urinalysis (via | X | X | | | | | | | | X | | X |
| dipstick) | | | | | | | | | | | | |
| Urine drug screen | X | X | | | | | | | | | | |
| Vital sign | X | X | X | | X | X | X | X | X | X | X | X |
| measurements | | | | | | | | | | | | |
| ECG | X | | Xⁱ | | | | | | | X | | |
| Cardiac telemetry^{j} | | | X | X | X | X | X | | | | | |
| Randomization^{k} | | | X | | | | | | | | | |
| Study drug | | | | X | | | | | | | | |
| administration | | | | | | | | | | | | |
| PK samples | | | X | | X | X | X | X | X | X | X | X |
| PD panel (serum | | | X | | X | X | X | X | X | X | X | X |
| C5, cRBC | | | | | | | | | | | | |
| hemolysis) | | | | | | | | | | | | |
| Infusion/injection | | | X | | | X | X | X | X | X | X | |
| site evaluation^{l} | | | | | | | | | | | | |
| Immunogenicity | | | X | | | | | | | | | |
| (ravulizumab | | | | | | | | | | | | |
| ADA) | | | | | | | | | | | | |

| | | | | |
|---|---|---|---|---|
| Review potential | X | X | X | X |
| safety risks of | | | | |
| ravulizumab^{m} | | | | |

| | |
|---|---|
| Concomitant | ←Monitor continuously (after ICF is signed at screening)→ |
| medications | |
| Adverse eventsⁿ | ←Monitor continuously (after ICF is signed at screening)→ |
| Prophylactic | ←Antibiotic prophylaxis→ |
| antibiotic tx^{o} | |

^{a}Permissible windows for study assessments are described in the study operations manual.
^{b}The EOI sample applies to the SC cohorts only and should be obtained within 10 min after the completion of SC infusion.
^{c}Subject will be discharged from the CRU after completing all Day 5 assessments. Subjects will be provided a “Study Participant ID card” with information for healthcare provider and subject on symptoms of meningitis infection.
^{d}Signed and dated EC-approved informed consent form (IFC) must be obtained before any study-specific screening procedures are performed.
^{e}For subjects who do not have adequate documentation of prior MCV4 immunization or serogroup B vaccination, MCV4 immunization is performed at least 56 days prior to dosing on Day 1, and vaccination for serogroup B meningococcal infections is administered at least 56 days prior to Day 1 dosing with a booster administered at least 28 days prior to dosing on Day 1.
^{f}For subjects with a documented vaccine titer within 6 months prior to screening, the titer does not need to be repeated.
^{g}Complement activity, confirmed by a suitable assay such as CAP ELISA/C5 (hemolysis) inhibition, is performed at screening to confirm subjects do not have a complement deficiency.
^{h}Serum pregnancy test for all female subjects of childbearing potential to confirm that a female subject is not pregnant prior to dosing.
ⁱOn Day 1, triplicate 12-lead electrocardiograms (ECGs) are performed predose and approximately 15 minutes after the EOI (Cohorts 1 through 4 only).
^{j}Continuous cardiac registration predose and until 3 hours after the SC injection (Cohorts 2, 3, and 4).
^{k}Planned randomization for Cohorts 1, 2, 3, and 5 may be up to 7 days prior to dosing on Day 1.
^{l}Injection site evaluations are performed within 15 minutes after EOI and ±15 minutes of the other scheduled times on Day 1.
^{m}The Investigator or qualified designee meets with the subject at each visit to discuss the potential safety risks of ravulizumab, and to address any safety concerns of the subject.
ⁿCollection of adverse events and serious adverse events begins after ICF signing.
^{o}Subjects are administered prophylactic antibiotic treatment, oral penicillin V 500 mg twice daily (equivalent to 1 × 106 units), beginning on the evening of Day −1 through Day 200, or until complement activity has normalized (as determined by CH50 assay).
Abbreviations:
ADA = antidrug antibody;
AE = adverse event;
BMI = body mass index;
CAP = complement alternative pathway;
cRBC = chicken red blood cell;
CRU = clinical research unit;
ECG = electrocardiogram;
EOI = end-of-infusion/injection;
h = hour;
HIV = human immunodeficiency virus;
ICF = informed consent form;
IV = intravenous;
MCV4 = tetravalent meningococcal conjugate vaccine;
min = minute;
OP = outpatient;
PD = pharmacodynamic(s);
PK = pharmacokinetic (s)
SC = subcutaneous;
SOI = start-of-infusion/injection;
TB = tuberculosis;
tx = treatment

TABLE 3
Schedule of Activities - Visit 2 Through Visit 14

| | Visit | Visit | Visit | Visit | Visit | Visit | Visit | Visit | Visit | Visit | Visit | Visit | Visit |
|---|---|---|---|---|---|---|---|---|---|---|---|---|---|
| | 2 | 3 | 4 | 5 | 6 | 7 | 8 | 9 | 10 | 11 | 12 | 13 | 14 |
| | Day 8 | Day | Day | Day | Day | Day | Day | Day | Day | Day | Day | Day | Day |
| Procedures^{a} | (168 h ± 2) | 15 | 22 | 29 | 36 | 43 | 50 | 57 | 71 | 92 | 120 | 150 | 200/ET |
| Status (OP or CRU) | OP | OP | OP | OP | OP | OP | OP | OP | OP | OP | OP | OP | OP |
| Physical examination | X | | | X | | | | | | | | | X |
| Vital sign | X | X | X | X | X | X | X | X | X | X | X | X | X |
| ECG | | | | | | | | | | | | X | X |
| Biochemistry | X | | | X | | | | X | | | | | X |
| Hematology | X | | | X | | | | X | | | | | X |
| Coagulation | X | | | X | | | | X | | | | | X |
| Urinalysis (via | X | | | X | | | | X | | | | | X |
| Serum pregnancy test | | | | | | | | X | | | X | | X |
| CH50 testing | | | | | | X | | X^{b} | | | | | |
| Pharmacokinetic | X | X | X | X | X | X | X | X | X | X | X | X | X |
| Pharmacodynamics | X | X | X | X | X | X | X | X | X | X | X | X | X |
| panel (serum C5, | | | | | | | | | | | | | |
| cRBC hemolysis) | | | | | | | | | | | | | |
| Immunogenicity | | X | | X | | | | X | | X | X | X | X |
| (ravulizumab ADA) | | | | | | | | | | | | | |

| | |
|---|---|
| Review potential | ←Discuss potential safety risks of ravulizumab→ |
| safety risks of | |
| ravulizumab^{c} | |
| Concomitant | ←Monitor continuously (after ICF is signed at screening)→ |
| medications | |
| Adverse events^{d} | ←Monitor continuously (after ICF is signed at screening)→ |
| Prophylactic antibiotic | ←Antibiotic prophylaxis→ |
| tx^{e} | |

^{a}Permissible windows for study assessments are described in the study operations manual.
^{b}Additional samples can be taken after Day 57 if complement has not normalized.
^{c}The Investigator or qualified designee meets with the subject at each visit to discuss the potential safety risks of ravulizumab, and to address any safety concerns on the part of the subject.
^{d}Collection of adverse events and serious adverse events begins after ICF signing.
^{e}Subjects are administered prophylactic antibiotic treatment, oral penicillin V 500 mg twice daily (equivalent to 1 × 106 units) through Day 200 or until complement activity has normalized (as determined by CH50 assay).
Abbreviations:
ADA = antidrug antibody;
cRBC = chicken red blood cell;
CRU = clinical research unit;
ECG = electrocardiogram;
ET = early termination;
h = hour;
ICF = informed consent form;
OP = outpatient;
tx = treatment

4. Inclusion Criteria

Subjects are eligible to be included in the study only if all of the following criteria apply:

A. Male or female subject must be at least 18 and 65 years of age, inclusive, at the time of signing the informed consent.

B. Body weight within 60-90 kg, inclusive, and body mass index within the range 18-29.9 kg/m², inclusive.

C. Negative serum pregnancy test at screening and Day −1.

D. Female subjects of childbearing potential and male subjects with female partners of childbearing potential must be willing to follow protocol-specified contraception guidance while on treatment and for up to 8 months after last dose of study drug.

E. QT interval corrected using the Fridericia's formula (QTcF)≤450 msec for male subjects and ≤470 msec for female subjects at screening and prior to dosing on Day 1.

F. Documented vaccination with MCV4 at least 56 days and not more than 2 years, 4 months prior to dosing. Documentation must include a positive titer to confirm an immune response before study drug administration.

G. Vaccination with serogroup B meningococcal vaccine at least 56 days prior to dosing on Day 1, with a booster administered at least 28 days prior to dosing on Day 1, with at least 28 days between the first and second injections.

H. Satisfactory medical assessment with no clinically significant or relevant abnormalities as determined by medical history, physical examination, vital signs, 12-lead ECG, and clinical laboratory evaluation (hematology, biochemistry, coagulation, and urinalysis) that is reasonably likely to interfere with the subject's participation in or ability to complete the study, or to potentially confound interpretation of study results, as assessed by the Investigator.

I. Willing and able to give written informed consent, which includes compliance with the requirements and restrictions listed in the informed consent form (ICF) and in this protocol.

5. Exclusion Criteria

Subjects are excluded from the study if any of the following criteria apply:

A. Current or recurrent disease (e.g., cardiovascular, hematological, neurological, endocrine, immunological, rheumatological, renal, hepatic or gastrointestinal or other conditions) that or could affect clinical assessments or clinical laboratory evaluations.

B. Current or relevant history of physical or psychiatric illness that are not stable or may require a change in treatment, use of prohibited therapies during the study or make the subject unlikely to fully comply with the requirements of the study or complete the study, or any condition that presents undue risk from the investigational product or study procedures.

C. Any other significant disease or disorder which, in the opinion of the Investigator, may put the subject at risk.

D. History of any Neisseria infection.

E. History of unexplained, recurrent infection, or infection requiring treatment with systemic antibiotics within 90 days prior to dosing on Day 1.

F. History of complement deficiency or complement activity below the reference range as evaluated at screening.

G. History of malignancy with the exception of a nonmelanoma skin cancer or carcinoma in-situ of the cervix that has been treated with no evidence of recurrence within 5 years.

H. Human immunodeficiency virus (HIV) infection (evidenced by HIV-1 or HIV-2 antibody titer).

I. Acute or chronic hepatitis B virus infection. Hepatitis B surface antigen (HBsAg) testing is required for all subjects prior to enrollment. Subjects with positive HBsAg will not be enrolled. For subjects with negative HBsAg, the following testing algorithm is required: If hepatitis B core antibody (HBcAb) is negative, the subject is eligible to enroll. If HBcAb is positive, the hepatitis B surface antibody (HBsAb) is tested. If both HBcAb and HBsAb are positive, the subject is eligible to enroll. If HBcAb is positive and HBsAb is negative, the subject is not enrolled.

J. Acute or chronic hepatitis C virus infection (evidenced by antibody titer).

K. Active systemic viral or fungal infection within 14 days prior to dosing.

L. History of latent or active tuberculosis (TB) or exposure to endemic areas within 8 weeks prior to the screening visit.

M. Documented history of allergy to penicillin or cephalosporin.

N. History of significant allergic reaction (e.g., anaphylaxis or angioedema) to any product (e.g., food, pharmaceutical).

O. Use of prescription medications (excluding oral contraceptives) within 14 days prior to dosing on Day 1, except with prior approval of the Sponsor.

P. Regular use of nonprescription, over-the-counter medications, including herbal remedies and supplements, within 14 days prior to dosing on Day 1. Multivitamins, paracetamol (acetaminophen) ≤2 g per day, and topical skin products without significant systemic absorption are allowed.

Q. Participation (i.e., last protocol-required study visit) in a clinical study within 90 days before initiation of dosing on Day 1.

R. Participation in more than 1 clinical study of a mAb, or participation (i.e., last protocol-required study visit) in a clinical study of a mAb within the 12 months prior to screening, during which the subject was exposed to the active study drug.

S. Positive or indeterminate QuantiFERON®-TB test indicating possible tuberculosis (TB) infection.

T. Presence of fever (confirmed body temperature >37.6° C.) (e.g., a fever associated with a symptomatic viral or bacterial infection) within 14 days prior to dosing on Day 1.

U. Serum creatinine greater than the upper limit of normal (ULN) of the reference range of the testing laboratory at screening or on Day −1.

V. Alanine aminotransferase (ALT) or aspartate aminotransferase (AST)>ULN of the reference range of the testing laboratory at screening or >1.5×ULN of the reference range of the testing laboratory on Day −1.

W. Any clinically significant abnormal hematological parameters (per the Investigator's discretion).

X. Positive urine drug toxicology screen at screening or on Day −1.

Y. Alcohol consumption within 48 hours prior to study drug administration or positive alcohol breath test on Day −1.

Z. Donation of plasma within 7 days prior to dosing on Day 1. Donation or loss (excluding volume drawn at screening) of more than 50 mL of blood within 30 days prior to dosing or more than 499 mL of blood within 56 days prior to dosing on Day 1.

AA. Female subjects who are breastfeeding.

BB. Subjects who are in intimate and prolonged contact with (defined as living under the same roof or providing personal care to) people younger than 2 years of age or older than 65 years of age, or who are either immunocompromised or have one of the following underlying medical conditions: anatomic or functional asplenia (including sickle cell disease); congenital complement, properdin, factor D, or primary antibody deficiencies; acquired complement deficiencies (e.g., those receiving eculizumab); or HIV.

CC. Subjects who are one of the following: professionals who are exposed to environments of greater risk for meningococcal disease, research, industrial, and clinical laboratory personnel who are routinely exposed to N meningitides, military personnel during recruit training (military personnel may be at increased risk of meningococcal infection when accommodated in close quarters), daycare center workers, those living on a college or university campus, or those who plan to travel during the course of the study to or have travelled to endemic areas for meningococcal meningitis (e.g., India, Sub-Saharan Africa, pilgrimage to Saudi Arabia for Hajj) within 6 months prior to dosing.

DD. Immunization with a live-attenuated vaccine 28 days prior to dosing on Day 1 or planned vaccination during the course of the study (except for the vaccination planned by the study protocol). Immunization with inactivated or recombinant influenza vaccine is permitted.

EE. Prior exposure to ravulizumab or eculizumab.

FF. Major surgery or hospitalization within 90 days prior to dosing on Day 1.

GG. History of allergy or hypersensitivity to excipients of ravulizumab (e.g., polysorbate 80), rHuPH20, or other hyaluronidases.

HH. Currently smokes >10 cigarettes daily (former smokers may be permitted to enroll at the Investigator's discretion) and is unwilling to refrain from smoking while a resident in the clinical research unit or comply with smoking restrictions.

II. History of illicit drug abuse, history of significant alcohol abuse within 1 year prior to the screening visit, or clinical evidence of substance and/or alcohol abuse within the 2 years before screening. Alcohol abuse is defined as regular weekly intake of more than 14 units (for both males and females), using the NHS alcohol tracker available at: nhs.uk/Tools/Pages/drinks-tracker.aspx. Study drug is defined as any investigational drug product(s), marketed product(s), or placebo, intended to be administered to a subject according to the protocol.

6. Study Drug

The study drug composition and doses administered in this study are presented in Table 4.

TABLE 4
Study Drug Compositions and Dose Reference Chart

| Study Drug | | | | Ravulizumab |
|---|---|---|---|---|
| Name: | Ravulizumab IV | Ravulizumab SC | rHuPH20 | SC/rHuPH20 |
| Dosage | Ravulizumab IV is | Ravulizumab | rHuPH20 is | Ravulizumab |
| Formulation: | formulated at pH | SC is formulated | supplied as | SC/rHuPH20 is |
| | 7.0 and each vial | at pH 7.4 and | ENHANZE ® | formulated per the |
| | contains 300 mg | each vial | drug product | individual |
| | of ravulizumab in | contains | (EDP) is supplied | components of the |
| | 10 mM sodium | 1100 mg of | in vials as a | drug product. |
| | phosphate, 150 | ravulizumab in | sterile, single- | |
| | mM sodium | 50 mM sodium | dose, injectable | |
| | chloride, 0.02% | phosphate, 25 | liquid at | |
| | polysorbate 80. | mM arginine, | approximately | |
| | Each vial contains | 5% sucrose, and | 110 kU/mL. The | |
| | 10 mg/mL. | 0.05% | solution has a pH | |
| | | polysorbate 80. | of 6.5 and | |
| | | Each vial | contains 130 mM | |
| | | contains 100 | sodium chloride, | |
| | | mg/mL. | 10 mM | |
| | | | L-Histidine/ | |
| | | | hydrochloride as | |
| | | | a buffer, 10 mM | |
| | | | L-Methionine, | |
| | | | and 0.2% w/w | |
| | | | polysorbate 80. | |
| | | | The solution is | |
| | | | filled to 0.5 mL | |
| | | | in a 2-mL glass | |
| | | | vial. | |
| Unit Dose | 400 mg | 400 mg | NA | ravulizumab SC |
| Strength(s)/ | | | | 500 mg/rHuPH20 |
| Dosage | | | | 10,000 units |
| Level(s): | | | | ravulizumab SC |
| | | | | 1000 mg/rHuPH20 |
| | | | | 20,000 units |
| | | | | ravulizumab SC |
| | | | | 2000 mg/rHuPH20 |
| | | | | 40,000 units |
| Route of | IV | SC | SC | SC |
| Administration | | | | |
| Dosing | A single dose of | A single dose of | NA | Ravulizumab is co- |
| Instructions: | ravulizumab IV is | ravulizumab SC | | mixed with EDP in |
| | administered via | is administered | | empty, sterile |
| | IV infusion and | via SC infusion | | 20-mL glass vials. |
| | 80 mL of | administered by | | A single dose of |
| | ravulizumab IV is | a syringe pump. | | ravulizumab |
| | administered at a | The total | | SC/rHuPH20 is |
| | maximum infusion | volume to be | | administered via |
| | rate of 333 mL/hr, | administered | | SC infusion, |
| | for a minimum | will be 4 mL at | | administered via an |
| | infusion duration | an infusion rate | | infusion pump. |
| | of approximately | of 0.5 mL/min. | | The total volume |
| | 15 minutes. | | | administered for |
| | Use of an in-line | | | the combination |
| | filter for infusion | | | cohorts is 5.23 mL |
| | is required. | | | in Cohort 2, |
| | | | | 10.46 mL in |
| | | | | Cohort 3, and |
| | | | | 20.91 mL in |
| | | | | Cohort 4 at an |
| | | | | infusion rate of |
| | | | | 2 mL/min. |
| Packaging and | Ravulizumab IV | Ravulizumab | rHuPH20 for | Individual |
| Labeling | drug product is | SC drug product | coadministration | components of |
| Additional | provided in a | is provided in a | is provided as | ravulizumab SC/ |
| instructions are | single-use, | single-use, | single-use, | rHuPH20 drug |
| provided in the | USP/Ph Eur | USP/Ph Eur | USP/Ph Eur | product are |
| pharmacy | Type 1 clear and | Type 1 clear and | Type I clear and | packaged and |
| manual. | colorless glass | colorless glass | colorless glass | labeled according |
| | vial, stoppered | vial, stoppered | vial. | to their respective |
| | with a gray butyl | with a gray butyl | | manufacturers. |
| | rubber stopper, | rubber stopper, | | |
| | and sealed with | and sealed with | | |
| | aluminum seal | aluminum seal | | |
| | with a | with a | | |
| | polypropylene | polypropylene | | |
| | flip-off cap. | flip-off cap. | | |
| Manufacturer | Alexion | Alexion | Halozyme | Alexion/Halozyme |

Abbreviations: IV = intravenous; NA = not applicable; SC = subcutaneous.
Source: rHuPH20 Investigator's Brochure (2018), pharmacy manual

The Investigator or designee confirms appropriate temperature conditions have been maintained during transit for all study drug received and that any discrepancies are reported and resolved before use of the study drug. Only subjects enrolled in the study receive study drug and only authorized site staff may supply or administer study drug. All study drugs are stored in a secure, environmentally controlled, and monitored (manually or automated) area in accordance with the labeled storage conditions with access limited to the Investigator and authorized site staff. Preparation of ravulizumab IV, ravulizumab SC, and the ravulizumab SC/rHuPH20 drug products is performed in accordance with local standards by qualified pharmacy personnel at the investigative site.

The handling and preparation of materials used to prepare and administer the study drug is carried out using aseptic techniques for sterile products. For each subject, doses are prepared as required per the dose cohort. The entire dosing apparatus (i.e., syringe and infusion tubing) is weighed before and after infusion and the weights recorded for the purpose of recoding the exact dose administered.

Recombinant human hyaluronidase PH20 is supplied as ENHANZE drug product (EDP; 1 mg/mL [0.5 mg of active ingredient per vial; approximately 110,000 units/mg]) in 2 mL single use glass vials.

The volume of drug product to be prepared is based on the cohort to which a subject is assigned. For Cohort 1, ravulizumab is administered undiluted via SC infusion administered by a syringe pump. For Cohorts 2 through 4, ravulizumab is co-mixed with EDP in empty, sterile glass vials. A single dose of ravulizumab SC/rHuPH20 is administered via SC infusion administered by a syringe pump. For Cohort 5, the IV admixture consists of ravulizumab diluted in a 1:1 ratio with 0.9% sodium chloride, Ph Eur, or BP. The IV infusion line is not flushed.

7. Concomitant Therapy

Subjects abstain from taking prescription or nonprescription drugs (including vitamins and dietary or herbal supplements) within 14 days before the start of study drug until completion of the follow-up visit, unless, in the opinion of the Investigator and Sponsor, the medication does not interfere with the study. Multivitamins, paracetamol (acetaminophen) (at doses of □2 g/day), and topical skin products without significant systemic absorption are permitted for use during the study at the Investigator's discretion. Topical skin products are not administered at the site of study drug injection from 24 hours prior until 24 hours following study drug administration. Subjects are also permitted to receive a booster vaccine, if required. Other concomitant medications are considered on a case-by-case basis by the Investigator in consultation with the medical monitor if required. Concomitant procedures are not allowed unless medically indicated.

8. Dose Modification

Decisions to continue, modify (explore the dose cohort further), or escalate dosing are made by the Investigator and/or SRC as described in Table 5. The SRC reviews all available safety and tolerability data from a given cohort for at least the first 168 hours after study drug administration to determine whether to escalate the ravulizumab SC/rHuPH20 dose and initiate the next cohort. Data through 168 hours must be available for at least 11 of the 12 subjects. Dose escalation or modification will occur based on the recommendation of the SRC to dose escalate and only applies to Cohort 3 and Cohort 4.

TABLE 5
Dose Continuation/Escalation Decision Pathway

| Dosing | Responsible | | Documentation/ |
|---|---|---|---|
| Decision | Party | Data to be Reviewed | Communication Methods |
| Continuation | Investigator | A minimum of 24 hours | The Investigator documents the |
| from the | | post-dose safety and | decision in an email to the |
| sentinel | | tolerability data from the | Sponsor. The email does not |
| subject to the | | sentinel subject. | require the Sponsor's response, |
| remaining | | | unless there is disagreement with |
| subjects in | | | the Investigator's decision. |
| Cohort 1 | | | |
| Continuation | Investigator | A minimum of 24 hours | The Investigator documents the |
| from the | | post-dose safety and | decision in an email to the |
| sentinel | | tolerability data from the | Sponsor. The email does not |
| subjects to the | | sentinel subjects. | require the Sponsor's response, |
| remaining | | | unless there is disagreement with |
| subjects in | | | the Investigator's decision. |
| Cohort 2 | | | |
| Escalation to | SRC | A minimum of 168 hours | The SRC documents the decision |
| Cohort 3 | | post-dose safety and | on the escalation/progression |
| | | tolerability data from at | approval form. |
| | | least 11 subjects (all of | |
| | | whom have received | |
| | | treatment) from the | |
| | | previous cohort that was | |
| | | on the next lowest dose | |
| | | level (and hence, next | |
| | | lowest exposure) of | |
| | | ravulizumab | |
| | | SC/rHuPH20. | |
| Continuation | Investigator | A minimum of 24 hours | The Investigator documents the |
| from the | | post-dose safety and | decision in an email to the |
| sentinel | | tolerability data from the | Sponsor. The email does not |
| subjects to the | | sentinel subjects. | require the Sponsor's response, |
| remaining | | | unless there is disagreement with |
| subjects in | | | the Investigator's decision. |
| Cohort 3 | | | |
| Escalation to | SRC | A minimum of 168 hours | The SRC documents the decision |
| Cohort 4 | | post-dose safety and | on the escalation/progression |
| | | tolerability data from at | approval form. |
| | | least 11 subjects (all of | |
| | | whom have received | |
| | | treatment) from the | |
| | | previous cohort that was | |
| | | on the next lowest dose | |
| | | level (and hence, next | |
| | | lowest exposure) of | |
| | | ravulizumab | |
| | | SC/rHuPH20. | |
| Continuation | Investigator | A minimum of 24 hours | The Investigator documents the |
| from the | | post-dose safety and | decision in an email to the |
| sentinel | | tolerability data from the | Sponsor. The email does not |
| subjects to the | | sentinel subjects. | require the Sponsor's response, |
| remaining | | | unless there is disagreement with |
| subjects in | | | the Investigator's decision. |
| Cohort 4 | | | |

Abbreviations: SC = subcutaneous; SRC = Safety Review Committee.

These rules apply to adverse events that are assessed as related to study drug by the Investigator. Dose continuation or escalation proceeds as scheduled (Table 3) and the study continues as planned, provided no prespecified toxicity events occur.

The entire study is suspended if any life-threatening (Common Terminology Criteria for Adverse Events [CTCAE] v4.03; published 14 Jun. 2010, Grade 4) or fatal (CTCAE Grade 5) SAEs occur. If any of the following occur, dosing within the affected cohort is suspended and dose escalation does not commence. Interim lower doses may be subsequently administered at the discretion of the SRC.

A treatment-related SAE, irrespective of the CTCAE grade, in 1 subject. This includes any subject potentially meeting the criteria for Hy's Law (ALT≥3×ULN) and bilirubin≥2×ULN (ie, >35% direct bilirubin) or ALT≥3×ULN and international normalized ratio (INR)>1.5, if INR was measured, which may indicate severe liver injury (possibly Hy's Law). Severe (CTCAE Grade 3) nonserious treatment-related AEs in 2 subjects in the same cohort, independent of whether the AEs are within the same System Organ Class (SOC).

For Cohorts 2 through 4 (ravulizumab SC/rHuPH20):
Any number of Grade 1 ISRs are permitted to allow continuation of dosing within a cohort and escalation to the next highest dose level.
Any number of Grade 2 ISRs that have resolved or reduced to Grade 1 by the time of the minimum data review period (168 hours post-dose) are permitted to allow continuation of dosing within a cohort and escalation to the next highest dose level.
If, at the end of the minimum data review period (168 hours post-dose) there are more than 2 subjects with ISRs that are still Grade 2, dose escalation does not occur and the period of observation is extended by a further 168 hours (or shorter, if all subjects recover to at least Grade 1 before that time point). If all affected subjects show signs of recovery (at least to Grade 1), dose escalation can proceed. If all affected subjects remain at Grade 2 after the additional 168-hour observation period, the SRC makes the decision to either prolong further the observation period or progress to subsequent combination cohorts at a lower dose/smaller volume of study drug.

9. Study Assessments and Procedures

All screening evaluations are completed and reviewed to confirm the subject meets all eligibility criteria. Planned time points for all safety assessments are presented in the Schedule of Activities (Tables 2 and 3).

Physical assessments include the following assessments: general appearance; skin; head, ears, eyes, nose, and throat; neck; lymph nodes; chest; heart; abdominal cavity; limbs; central nervous system; and musculoskeletal system. Height and weight (screening only) are also measured and recorded. Body mass index is calculated and recorded at screening.

Vital sign measurements are taken after the subject has been resting in the supine or semi-recumbent position for at least 5 minutes and include temperature (° C.; oral), respiratory rate, supine blood pressure, and pulse. The timing of vital sign measurements is described in the Schedule of Activities (Tables 2 and 3). Out of range blood pressure or heart rate measurements are repeated at the Investigator's discretion. Confirmed, clinically significant vital sign measurements are recorded as adverse events.

A triplicate 12-lead electrocardiogram (ECG) is obtained after the subject has been resting for at least 5 minutes. The timing of ECGs is described in the Schedule of Activities. At each time point at which triplicate ECGs are required, 3 individual ECG tracings are obtained as closely as possible in succession, but no more than 2 minutes apart. The full set of triplicates are completed in less than 4 minutes, 30 seconds. In addition, continuous cardiac registration is performed in Cohorts 2, 3, and 4.

All protocol-required laboratory assessments are conducted in accordance with the Schedule of Activities and the laboratory manual. Clinical and laboratory assessments are performed by a local laboratory to assess safety of ravulizumab. The Investigator reviews the laboratory report, documents this review, and records all clinically relevant changes occurring during the study in the adverse event (AE) section of the electronic case report form (eCRF). The laboratory reports must be filed with the source documents. All laboratory tests with values considered clinically significantly abnormal during participation in the study are repeated until the values return to normal or baseline or are no longer considered clinically significant by the Investigator or medical monitor. If such values do not return to normal/baseline within a period of time judged reasonable by the Investigator, the etiology is identified and the Sponsor is notified. If laboratory values from non-protocol-specified laboratory assessments performed at the institution's local laboratory require a change in subject management or are considered clinically significant by the Investigator (e.g., serious adverse event, adverse event, or dose modification), then the results are recorded. The maximum amount of blood collected from each subject over the duration of the study, including any extra assessments that may be required, does not exceed 500 mL. Repeat or unscheduled samples can be obtained for safety and/or eligibility reasons or if there are any technical issues with the samples.

Blood samples collected at screening are analyzed for HIV-1, HIV-2, HBsAg, and hepatitis C virus antibody titers. Hepatitis B surface antigen testing is required for all subjects prior to enrollment. Subjects with positive HBsAg are not enrolled. A titer against meningococcal serogroups A, C, W135, and Y is performed at screening. Titer measurements are used to exclude subjects without a confirmed immune response.

Antibodies to ravulizumab are evaluated in serum samples collected from all subjects according to the Schedule of Activities. Serum samples are screened for antibodies that bind to ravulizumab and the titer of confirmed positive samples is reported. The detection and characterization of antibodies to ravulizumab is performed using a validated assay method by or under the supervision of the Sponsor. Samples can be banked for a period of up to 5 years in order to perform additional safety assessments, as necessary.

A urine sample for drug screen is analyzed for substances. Timing of urine drug and alcohol breath tests is specified in the Schedule of Activities.

Pregnancy testing are performed for all female subjects at the time points specified in the Schedule of Activities.

Serum samples for a QuantiFERON-TB test are obtained at the time points specified in the Schedule of Activities.

Subcutaneous injection or IV infusion-site evaluations is performed at the time points specified in the Schedule of Activities. Injection site reactions (e.g., indurations ≤1 cm in size) are not listed as an adverse event unless they persist for more than 24 hours.

To mitigate the risk of N meningitidis infection associated with terminal complement inhibition, subjects in this study are administered the following:

1. A MCV4 vaccination at least 56 days prior to dosing of ravulizumab on Day 1 (if not vaccinated with MCV4 within the last 3 years, or if subjects have been previously vaccinated but there is not adequate documentation to verify prior vaccination).

2. Two injections of the serogroup B meningococcal vaccine. The first injection must be administered at least 56 days prior to dosing on Day 1, with a booster administered at least 28 days prior to dosing on Day 1, with at least 28 days between the first and second injections.

3. Prophylactic antibiotic treatment, oral penicillin V 500 mg twice daily (equivalent to 1×10⁶ units) until complement activity has normalized (as determined by CH50 assay).

The first dose of antibiotic is administered orally on Day −1 in the evening, prior to the Day 1 (dose administration) of study drug. For the outpatient portion of the study, subjects are instructed to take the antibiotic approximately at the same times (twice daily) on each scheduled day. A suitable system (such as text messaging) is used for daily monitoring of subjects' compliance with the antibiotic prophylaxis regimen.

The following observations support the administration of antibiotic prophylaxis in this single-dose study: Penicillin is the drug of choice in eradication of N meningitidis in carriers. Complement-deficient patients who received monthly injections with benzathine penicillin G as prophylaxis for recurrent meningococcal disease during a 2- to 4-year period experienced significantly fewer episodes of Neisseria infection than deficient individuals not receiving prophylaxis (Figueroa J E, et al., Clin Microbiol Rev. 1991 July; 4(3):359-395). High levels of resistance to penicillin caused by plasmid-encoded β-lactamases are rarely encountered in meningococcal strains (Yazdankhah S P, et al., J Med Microbiol. 2004 September; 53(Pt 9):821-832). 1. Antibiotic prophylaxis with orally administered penicillin V 500 mg twice daily has been provided in the treatment of PNH and aHUS patients with eculizumab by some physicians and is generally well-tolerated (Kelly R J, et al., Blood. 2011 Jun. 23; 117(25):6786-6792 and Leeds Teaching Hospitals NHS Trust, Kings College Hospital NHS Foundation Trust. National Specialised Commissioning Team (NSCT) Service Specification Paroxysmal Nocturnal Haemoglobinuria (PNH). 2013).

Risk of infection is explained and discussed with subjects during the informed consent process, occurring at the screening visit. In order to increase the risk awareness and promote quick disclosure of any potential signs or symptoms of infection experienced by the subjects during the course of the study, additional discussion and explanation of the potential risks, signs, and symptoms, as described in the informed consent form, take place at specific time points throughout the study as noted in the Schedule of Activities (Tables 2 and 3). Subjects are also provided a safety card to carry with them at all times.

Adverse events (AE) are reported to the Investigator or qualified designee by the subject (or, when appropriate, by a caregiver, surrogate, or the subject's legally authorized representative). An adverse event is any untoward medical occurrence in a patient or clinical study subject, temporally associated with the use of study drug, whether or not considered related to the study drug. An adverse event can therefore, be any unfavorable and unintended sign (including an abnormal laboratory finding), symptom, or disease (new or exacerbated) temporally associated with the use of study drug.

Events that meet the adverse event definition include: any abnormal laboratory test results (hematology, clinical chemistry, or urinalysis) or other safety assessments (e.g., electrocardiogram, radiological scans, vital signs measurements), including those that worsen from baseline, considered clinically significant in the medical and scientific judgment of the Investigator (i.e., not related to progression of underlying disease), new conditions detected or diagnosed after study drug administration even though it may have been present before the start of the study, or signs, symptoms, or the clinical sequelae of a suspected drug-drug interaction.

Events that do not meet the adverse event definition are medical or surgical procedure (e.g., endoscopy, appendectomy): the condition that leads to the procedure is the adverse event, situations in which an untoward medical occurrence did not occur (social and/or convenience admission to a hospital), anticipated day-to-day fluctuations of pre-existing disease(s) or condition(s) present or detected at the start of the study that do not worsen.

If an event is not an adverse event per definition above, then it cannot be a serious adverse event (SAE) even if serious conditions are met (e.g., hospitalization for signs/symptoms of the disease under study, death due to progression of disease). A serious adverse event is defined as any untoward medical occurrence that, at any dose:
a. Results in death.
b. Is life-threatening. The term ‘life-threatening’ in the definition of ‘serious’ refers to an event in which the subject was at risk of death at the time of the event. It does not refer to an event, which hypothetically might have caused death, if it were more severe.
c. Requires inpatient hospitalization or prolongation of existing hospitalization. In general, hospitalization signifies that the subject has been detained (usually involving at least an overnight stay) at the hospital or emergency ward for observation and/or treatment that would not have been appropriate in the physician's office or outpatient setting. Complications that occur during hospitalization are adverse events. If a complication prolongs hospitalization or fulfills any other serious criteria, the event is serious. When in doubt as to whether “hospitalization” occurred or was necessary, the adverse event should be considered serious. Hospitalization for elective treatment of a pre-existing condition that did not worsen from baseline is not considered an adverse event.
d. Results in persistent disability/incapacity. The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.
e. Is a congenital anomaly/birth defect.
f. Other situations: Medical or scientific judgment is exercised in deciding whether serious adverse event reporting is appropriate in other situations, such as important medical events that may not be immediately life-threatening or result in death or hospitalization but may jeopardize the subject or may require medical or surgical intervention to prevent one of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

When an adverse event or serious adverse event occurs, it is the responsibility of the Investigator to review all documentation (e.g., hospital progress notes, laboratory reports, and diagnostics reports) related to the event. The Investigator records all relevant adverse event or serious adverse event information. The Investigator makes an assessment of intensity for each adverse event and serious adverse event reported during the study and assigns it to one of the following categories from National Cancer Institute CTCAE v4.03 (published 14 Jun. 2010): Grade 1: Mild (awareness of sign or symptom, but easily tolerated), Grade 2: Moderate (discomfort sufficient to cause interference with normal activities), Grade 3: Severe (incapacitating, with inability to perform normal activities), Grade 4: Life-threatening, or Grade 5: Fatal. Changes in the severity of an adverse event should be documented to allow an assessment of the adverse event duration at each level of intensity evaluated. Adverse events characterized as intermittent require documentation of onset and duration of each episode, if the severity of the intermittent event changes. An event is defined as ‘serious’ when it meets at least one of the predefined outcomes as described in the definition of an serious adverse event, not when it is rated as severe.

The Investigator is obligated to assess the relationship between study drug and each occurrence of each adverse event/serious adverse event. An Investigator causality assessment is provided for all adverse events (both nonserious and serious). This assessment is recorded in the data capture system and on any additional forms, as appropriate. The definitions for the causality assessments are as follows:

Not related (unrelated): This relationship suggests that there is no association between the investigational product and the reported event.

Unlikely related: This relationship suggests that the clinical picture is highly consistent with a cause other than the investigational product, but attribution cannot be made with absolute certainty, and a relationship between the investigational product and adverse event cannot be excluded with complete confidence.

Possibly related: This relationship suggests that treatment with the investigational product may have caused or contributed to the adverse event, i.e., the event follows a reasonable temporal sequence from the time of study drug administration, and/or follows a known response pattern to the investigational product, but could also have been produced by other factors.

Probably related: This relationship suggests that a reasonable temporal sequence of the event with the investigational product administration exists, as well as the likely association of the event with the investigational product. This is based upon the known pharmacological action of the investigational product, known or previously reported adverse reactions to the investigational product or class of drugs, or judgment based on the Investigator's clinical experience.

Definitely related: Temporal relationship to the investigational product. Other conditions (concurrent illness, concurrent medication reaction, or progression/expression of disease state) do not appear to explain the event; the event corresponds with the known pharmaceutical profile; improvement on discontinuation; reappearance on rechallenge.

The Investigator uses clinical judgment to determine the relationship. Alternative causes, such as underlying disease(s), concomitant therapy, and other risk factors, as well as the temporal relationship of the event to study drug administration are considered and investigated.

Infusion of other monoclonal antibodies has been associated with infusion reactions, with onset typically during or shortly after completion of the infusion. For this reason, subjects are carefully observed during each infusion. Subjects are closely monitored during and after study drug administration for any symptoms of anaphylaxis and other hypersensitivity reactions, including circulatory and/or respiratory changes or arrest, or urticaria, arthralgias, myalgias, or other signs of related reactions. Adequate treatment is immediately available. Infusion-associated adverse events may occur, and depending on their type and severity, discontinuation of infusion may be required. Subjects are informed of early symptoms and signs of hypersensitivity reactions including hives, swollen face, eyelids, lips, or tongue, or trouble with breathing. An acute infusion reaction algorithm is used to manage infusion-related reactions. In this study, regular assessments to monitor infusion reactions and infusion-site reactions are done. To ensure that reactions can be dealt with promptly, there is at least 15 minutes between the end of IV/SC infusion in 1 subject and the start of IV/SC infusion in the next subject. No more than 6 subjects assigned to receive ravulizumab IV are dosed per day. Any reactions are treated and taken into account in the dose continuation/escalation and toxicity rules. If anaphylactic reactions occur, the current “UK Treatment Guideline for Anaphylactic Reactions” of the UK Resuscitation Council are followed.

Subjects who experience a severe reaction during administration of study drug that results in discontinuation of study drug undergo all scheduled safety, immunogenicity, PK, and PD evaluations required by the protocol. Subjects are therefore be instructed to attend all scheduled visits and undergo all procedures per protocol.

Infusion-site reactions are defined as adverse events localized to the site of IV or SC route of study drug administration, occurring at any time during study participation that are assessed by the Investigator to be possibly, probably, or definitely related to study drug. Infusion-associated reactions are defined as systemic adverse events (e.g., fever, chills, flushing, alterations in heart rate and blood pressure, dyspnea, nausea, vomiting, diarrhea, and generalized skin rashes) occurring during or within 24 hours of the start of IV or SC infusion that are assessed by the Investigator to be possibly, probably, or definitely related to the study drug. No cases of overdose have been reported during ravulizumab IV or SC clinical studies. A single dose of study drug is administered and monitored by site personnel.

Whole blood samples are collected for measurement of serum concentrations of study drug as specified in the Schedule of Activities (Tables 2 and 3). Additional samples can be collected during the study if warranted and agreed upon between the Investigator and the Sponsor. The actual date and time (24-hour clock time) of each sample is recorded. Additional details, including further handling and processing instructions and sampling time windows are provided in the study laboratory manual.

After study drug administration, whole blood samples are collected for measurement of serum free C5 concentrations, chicken red blood cell (cRBC) hemolytic activity, and potentially other measures of C5 activation as specified in the Schedule of Activities (Tables 1 and 2). Additional samples can be collected during the study if warranted and agreed upon between the Investigator and the Sponsor.

Serum samples are collected at baseline and during follow-up for measurement of CH50 activity using an in vitro liposome immunoassay (LIA) to confirm normalization of complement activity. If a normal CH50 result is obtained from a subject's first CH50 sample collected during follow-up, antibiotic prophylaxis is stopped and the second scheduled CH50 sample is not required. If the first and second CH50 samples are not normal, the baseline sample can be analyzed, and further CH50 samples are taken until complement activity has been normalized.

10. Statistical Methods and Analyses

The sample size is based on pharmacokinetic rather than statistical considerations. A total sample size of 48 subjects (6 subjects each in the control cohorts [Cohort 1 and 5] and 12 subjects each in the combination cohorts [Cohorts 2, 3, and 4]) serve to estimate bioavailability.

TABLE 6
Analysis Sets

| Set | Description |
|---|---|
| Safety | All subjects who receive at least 1 dose of study |
| | drug |
| Pharmacokinetic | All subjects who have sufficient serum concentra- |
| | tion data to enable the calculation of PK parameters |
| Pharmacodynamic | All subjects who have sufficient total and free C5 |
| | concentration data and cRBC hemolysis data which |
| | will enable the evaluation of the PD effects |
| Immunogenicity | All subjects who have a predose and post-dose ADA |
| | sample collected |

Abbreviations: ADA = antidrug antibody; C5 = complement component 5; cRBC = chicken red blood cell; PD = pharmacodynamic(s); PK = pharmacokinetic(s).

In general, descriptive statistics for continuous variables include number of non-missing values, arithmetic mean, standard deviation, median, minimum, and maximum. Descriptive statistics for PK parameters include number of observations, arithmetic mean, standard deviation, arithmetic coefficient of variation (% CV), median, minimum, maximum, geometric mean and geometric % CV. Categorical variables are summarized using percentages and frequency counts, by cohort and time point.

A statistical analysis plan (SAP) is developed and finalized before data cutoff/database lock and further describes the subject populations to be included in the analyses, and procedures for accounting for missing, unused, and spurious data as appropriate. This section is a high-level summary of the planned statistical analyses of the primary and secondary endpoints.

No efficacy analyses are performed for this study. All safety analyses are performed on the Safety Set and reported by each cohort. Safety analyses include an analysis of all adverse events, electrocardiograms, clinical laboratory data, physical examinations, and vital sign measurements using descriptive statistics. No inferential statistical analyses are planned on the safety parameters of this study. The incidence of adverse events and serious adverse events is summarized, by system organ class (SOC) and Preferred Term for each cohort and overall, by relationship to study drug. Adverse events are also summarized by cohort and overall by severity. Serious adverse events and adverse events resulting in withdrawal from the study are listed. Subjects having multiple adverse events within a category (e.g., overall, system organ class, Preferred Term) are counted once in that category. For severity tables, a subject's most severe event within a category is counted.

Changes from baseline in vital sign measurements and laboratory assessments (e.g., chemistry, cell blood count with differential, and urinalysis) are summarized by each cohort. Laboratory parameter values are graded according to the Common Terminology Criteria for Adverse Events (CTCAE). Shift tables by cohort are produced for these laboratory parameters. These tables summarize the number of subjects with each baseline grade relative to the reference ranges and changes to the worst highest grade assessed post-dose during the study.

The ECG parameters are measured at the specified time points, including heart rate, PR, RR, QRS, QT, and corrected QTcF intervals. The average of the triplicate ECG readings at the time points collected is calculated, and changes from pretreatment baseline values are assessed by each cohort.

An outlier analysis is performed that summarizes the frequency and percentage of subjects who meet any of the following outlier criteria at each visit by cohort: QT, QTcF interval >450 msec; QT, QTcF interval >480 msec; QT, QTcF interval >500 msec; QT, QTcF interval increases from baseline >30 msec; and QT, QTcF interval increases from baseline >60 msec.

All concomitant medications are coded using the World Health Organization Drug Dictionary, and the frequency and percentage of concomitant medications is summarized. The individual serum concentration data for ravulizumab IV-, ravulizumab SC/rHuPH20-, ravulizumab SC-treated subjects, with actual sampling dates and times, is used to derive the pharmacokinetic parameters by noncompartmental analyses methods using Phoenix WinNonlin 6.3 or higher. The following PK parameters are derived: maximum observed serum concentration (Cₘₐₓ), time to maximum observed serum concentration (tmax), area under the serum concentration versus time curve from time 0 to the last quantifiable concentration (AUCₜ), area under the curve from time 0 (dosing) to time infinity (AUC_{0-∞}), apparent terminal-phase elimination rate constant (λ_{z}), terminal elimination half-life (t_{1/2}), total clearance (CL) or apparent clearance (CL/F), volume of distribution (V_{d}) or apparent volume of distribution (V_{d}/F), absolute bioavailability (F), and relative bioavailability (Fᵣₑₗ). The absolute bioavailability for the ravulizumab SC/rHuPH20 cohorts is defined by the ratio of the geometric means for the AUC_{0-∞} parameter for the ravulizumab SC/rHuPH20 cohort over the ravulizumab IV cohort. The relative bioavailability for the ravulizumab SC/rHuPH20 cohorts is defined by the ratio of the geometric means for the AUC_{0-∞} parameter for the ravulizumab SC/rHuPH20 cohort over the ravulizumab SC cohort. For the absolute and relative bioavailability estimates, a 95% CI for each of the ratio of the geometric means is provided.

The pharmacodynamic effects of ravulizumab SC and IV are evaluated by assessing changes in serum free C5 concentrations, cRBC hemolysis, and other measures of C5 activation over time as appropriate.

Immunogenicity, as measured by antidrug antibody, is summarized for ravulizumab.

SEQUENCE SUMMARY
SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSS
SEQ ID NO: 8
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK
SEQ ID NO: 9
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
SEQ ID NO: 10
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 11
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 12
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGV
HTFPAVLQSSGLYSLSSVVTVTSSNFGTQTYTCNVDHKPSNTKVDKTVER
KCCVECPPCPAPPVAGPSVFLFPPKPKDTLYITREPEVTCVVVDVSHEDP
EVQFNWYVDGMEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
SEQ ID NO: 16
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVTSSNFGT
QTYTCNVDHKPSNTKVDKTVERKCCVECPPCPAPPVAGPSVFLFPPKPKD
TLYITREPEVTCVVVDVSHEDPEVQFNWYVDGMEVHNAKTKPREEQFNST
FRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLD
SDGSFFLYSKLTVDKSRWQQGNVFSCSVMHEALHNHYTQKSLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGG
IGPFFGTANYAQKFQGRVTITADESTSTAYMELSSLRSEDTAVYYCARDT
PYFDYWGQGTLVTVSS
SEQ ID NO: 28
DIELTQPPSVSVAPGQTARISCSGDSIPNYYVYWYQQKPGQAPVLVIYDD
SNRPSGIPERFSGSNSGNTATLTISGTQAEDEADYYCQSFDSSLNAEVFG
GGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGII
DPDDSYTEYSPSFQGQVTISADKSISTAYLQWSSLKASDTAMYYCARYEY
GGFDIWGQGTLVTVSS
SEQ ID NO: 36
SYELTQPPSVSVAPGQTARISCSGDNIGNSYVHWYQQKPGQAPVLVIYKD
NDRPSGIPERFSGSNSGNTATLTISGTQAEDEADYYCGTYDIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWVG
AIYTGSGATYKASWAKGRFTISKDTSKNQVVLTMTNMDPVDTATYYCASD
GGYDYPTHAMHYWGQGTLVTVSS
SEQ ID NO: 44
DVVMTQSPSSLSASVGDRVTITCQASQNIGSSLAWYQQKPGQAPRLLIYG
ASKTHSGVPSRFSGSGSGTDFTLTISSLQPEDVATYYCQSTKVGSSYGNH
FGGGTKVEIK
SEQ ID NO: 45
QVQLVESGGGLVQPGRSLRLSCAASGFTVHSSYYMAWVRQAPGKGLEWVG
AIFTGSGAEYKAEWAKGRVTISKDTSKNQVVLTMTNMDPVDTATYYCASD
AGYDYPTHAMHYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELRRGPKVFLFP
PKPKDTLMISRTPEVTCVVVDVSHEDPEVKFNWYVDGVEVHNAKTKPREE
QYNSTYRVVSVLTVLHQDWLNGKEYKCKVSNKGLPSSIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVLHEALHAHYTRKELSLS
P
SEQ ID NO: 46
DIQMTQSPSSLSASVGDRVTITCRASQGISSSLAWYQQKPGKAPKLLIYG
ASETESGVPSRFSGSGSGTDFTLTISSLQPEDFATYYCQNTKVGSSYGNT
FGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
SEQ ID NO: 47
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSS
SEQ ID NO: 48
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIK
SEQ ID NO: 49
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLEPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYA
ASSLQSGVPSRFAGRGSGTDFILTISSLQPEDFATYYCLQDFNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 51
MGVLKFKHIFFRSFVKSSGVSQIVFTFLLIPCCLTLNFRAPPVIPNVPFL
WAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTEATEKAKQEFEKAGKDFL
VETIKLGKLLRPNHLWGYYLFPDCYNHHYKKPGYNGSCFNVEIKRNDDLS
WLWNESTALYPSIYLNTQQSPVAATLYVRNRVREAIRVSKIPDAKSPLPV
FAYTRIVFTDQVLKFLSQDELVYTFGETVALGASGIVIWGTLSIMRSMKS
CULDNYMETILNPYIINVTLAAKMCSQVLCQEQGVCIRKNWNSSDYLHLN
PDNFAIQLEKGGKFTVRGKPTLEDLEQFSEKFYCSCYSTLSCKEKADVKD
TDAVDVCIADGVCIDAFLKPPMETEEPQIFYNASPSTLSATMFIVSILFL
IISSVASL
SEQ ID NO: 52
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASG
IVIWGTLSIMRSMKSCLLLDNYMETILNPYIINVTLAAKMCSQVLCQEQG
VCIRKNWNSSDYLHLNPDNFATQLEKGGKFTVRGKPTLEDLEQFSEKEYC
SCYSTLSCKEKADVKDTDAVDVCIADGVCIDAFLKPPMETEEPQIFYN
SEQ ID NO: 53
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGHLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASG
IVIWGTLSIMRSMKSCLLLDNYMETILNPYIINVTLAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDAFLKPPMETEEPQIFY
SEQ ID NO: 54
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFY
MPVDNLGMAVIDWEEWRPTWARNWKPKDVYKNRSIELVQQQNVQLSLTEA
TEKAKQEFEKAGKDFLVETIKLGKLLRPNHLWGYYLFPDCYNHHYKKPGY
NGSCFNVEIKRNDDLSWLWNESTALYPSIYLNTQQSPVAATLYVRNRVRE
AIRVSKIPDAKSPLPVFAYTRIVETDQVLKELSQDELVYTFGETVALGAS
GIVIWGTLSIMRSMKSCLLLDNYMETILNPYIINVTLAAKMCSQVLCQEQ
GVCIRKNWNSSDYLHLNPDNFAIQLEKGGKFTVRGKPTLEDLEQFSEKFY
CSCYSTLSCKEKADVKDTDAVDVCIADGVCIDAFLKPPMETEEPQIF
SEQ ID NO: 55
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVETDQVLKELSQDELVYTFGETVALGASG
IVIWGTLSIMRSMKSCLLLDNYMETILNPYIINVILAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDAFLKPPMETEEPQI
SEQ ID NO: 56
LNFRAPPVIPNVPFLWAWNAPSEFCLGKEDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNIQQSPVAATLYVRNRVREA
IRVSKIPDAKSPLPVFAYTRIVFIDQVLKFLSQDELVYTFGETVALGASG
IVIWGILSIMRSMKSCLLLDNYMETILNPYIINVTLAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDAFLKPPMETEEPQ
SEQ ID NO: 57
LNFRAPPVIPNVPFLWAWNAPSEFCLGKEDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFIDQVLKFLSQDELVYTFGETVALGASG
IVIWGILSIMRSMKSCLLLDNYMETILNPYIINVTLAAKMCSQVLCQEQG
VCIRKNWNSSDYLHLNPDNFAIQLEKGGKFTVRGKPTLEDLEQFSEKEYC
SCYSTLSCKEKADVKDTDAVDVCIADGVCIDAFLKPPMETEEP
SEQ ID NO: 58
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASG
IVIWGTLSIMRSMKSCLLLDNYMETILNPYIINVTLAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDAFLKPPMETEE
SEQ ID NO: 59
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASG
IVIWGTLSIMRSMKSCLLLDNYMETILNPYIINVTLAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDA
SEQ ID NO: 60
LNFRAPPVIPNVPFLWAWNAPSEFCLGKFDEPLDMSLFSFIGSPRINATG
QGVTIFYVDRLGYYPYIDSITGVTVNGGIPQKISLQDHLDKAKKDITFYM
PVDNLGMAVIDWEEWRPTWARNWKPKDVYKNRSIELVQQQNVQLSLTEAT
EKAKQEFEKAGKDFLVETIKLGKLLRPNHLWGYYLFPDCYNHHYKKPGYN
GSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASG
IVIWGTLSIMRSMKSCLLLDNYMETILNPYIINVTLAAKMCSQVLCQEQG
VCIRKNWNSSDYLHLNPDNFAIQLEKGGKFTVRGKPTLEDLEQFSEKFYC
SCYSTLSCKEKADVKDTDAVDVCIADGVCIDAFLKPPMETEEPQIFYN
SEQ ID NO: 61
atgggagtgc taaaattcaa gcacatcttt ttcagaagct
ttgttaaatc aagtggagta tcccagatag ttttcacctt
ccttctgatt ccatgttgct tgactctgaa tttcagagca
cctcctgtta ttccaaatgt gcctttcctc tgggcctgga
atgccccaag tgaattttgt cttggaaaat ttgatgagcc
actagatatg agcctcttct ctttcatagg aagcccccga
ataaacgcca ccgggcaagg tgttacaata ttttatgttg
atagacttgg ctactatcct 3 tacatagatt caatcacagg
agtaactgtg aatggaggaa tcccccagaa gatttcctta
caagaccatc tggacaaagc taagaaagac attacatttt
atatgccagt agacaatttg ggaatggctg ttattgactg
ggaagaatgg agacccactt gggcaagaaa ctggaaacct
aaagatgttt acaagaatag gtctattgaa ttggttcagc
aacaaaatgt acaacttagt ctcacagagg ccactgagaa
agcaaaacaa gaatttgaaa aggcagggaa ggatttcctg
gtagagacta taaaattggg aaaattactt cggccaaatc
acttgtgggg ttattatctt tttccggatt gttacaacca
tcactataag aaacccggtt acaatggaag ttgcttcaat
gtagaaataa aaagaaatga tgatctcagc tggttgtgga
atgaaagcac tgctatttac ccatccattt atttgaacac
tcagcagtct cctgtagctg ctacactcta tgtgcgcaat
cgagttcggg aagccatcag agtttccaaa atacctgatg
caaaaagtcc acttccggtt tttgcatata cccgcatagt
ttttactgat caagttttga aattcctttc tcaagatgaa
cttgtgtata catttggcga aactgttgct ctgggtgctt
ctggaattgt aatatgggga accctcagta taatgcgaag
tatgaaatct tgcttgctcc tagacaatta catggagact
atactgaatc cttacataat caacgtcaca ctagcagcca
aaatgtgtag ccaagtgctt tgccaggagc aaggagtgtg
tataaggaaa aactggaatt caagtgacta tcttcacctc
aacccagata attttgctat tcaacttgag aaaggtggaa
agttcacagt acgtggaaaa ccgacacttg aagacctgga
gcaattttct gaaaaatttt attgcagctg ttatagcacc
ttgagttgta aggagaaagc tgatgtaaaa gacactgatg
ctgttgatgt gtgtattgct gatggtgtct gtatagatgc
ttttctaaaa cctcccatgg agacagaaga acctcaaatt
ttctac

What is claimed is:

1. A method of treating a human patient with a complement-associated condition, the method comprising subcutaneously co-administering to the patient a hyaluronidase and an anti-C5 antibody, or antigen binding fragment thereof, wherein the anti-C5 antibody, or antigen binding fragment thereof, comprises CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively.

2. The method of claim 1, wherein the anti-C5 antibody, or antigen binding fragment thereof, further comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

3. The method of claim 1 or 2, wherein the anti-C5 antibody, or antigen-binding fragment thereof, comprises a heavy chain variable region set forth in SEQ ID NO:12 and a light chain variable region set forth in SEQ ID NO:8.

4. The method of any one of the preceding claims, wherein the anti-C5 antibody, or antigen-binding fragment thereof, further comprises a heavy chain constant region set forth in SEQ ID NO:13.

5. The method of any one of the preceding claims, wherein the antibody, or antigen-binding fragment thereof, comprises a heavy chain polypeptide comprising the amino acid sequence set forth in SEQ ID NO:14 and a light chain polypeptide comprising the amino acid sequence set forth in SEQ ID NO:11.

6. The method of any one of the preceding claims, wherein the anti-C5 antibody, or antigen-binding fragment thereof, binds to human C5 at pH 7.4 and 25° C. with an affinity dissociation constant (K_{D}) that is in the range 0.1 nM≤K_{D}≤1 nM.

7. The method of any one of the preceding claims, wherein the anti-C5 antibody, or antigen-binding fragment thereof, binds to human C5 at pH 6.0 and 25° C. with a K_{D}≥10 nM.

8. The method of any one of the preceding claims, wherein the antibody is ravulizumab.

9. The method of any one of the preceding claims, wherein the antibody, or antigen-binding fragment thereof, is ravulizumab administered in a formulation comprising 1100 mg of ravulizumab, 50 mM sodium phosphate, 25 mM arginine, 5% sucrose, and 0.05% polysorbate 80.

10. The method of any one of the preceding claims, wherein the hyaluronidase is a recombinant human hyaluronidase.

11. The method of claim 10, wherein the recombinant human hyaluronidase comprises the amino acid sequence set forth in any one of SEQ ID NOs:51-60.

12. The method of claim 10, wherein the recombinant human hyaluronidase is rHuPH20.

13. The method of claim 10, wherein the recombinant human hyaluronidase is rHuPH20 administered in a formulation comprising approximately 110 kU/mL of rHuPH20, 130 mM sodium chloride, 10 mM L-Histidine/hydrochloride, 10 mM L-Methionine and 0.2% w/w polysorbate 80.

14. The method of claim 13, wherein the rHuPH20 formulation is ENHANZE®.

15. The method of any one of the preceding claims, wherein the hyaluronidase and antibody, or antigen-binding fragment thereof, are administered simultaneously in separate formulations.

16. The method of any one of claims 1-14, wherein the hyaluronidase and antibody, or antigen-binding fragment thereof, are mixed and administered in a single formulation.

17. The method of any one of the preceding claims, wherein the hyaluronidase is rHuPH20 (ENHANZE®) administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

18. The method of any one of the preceding claims, wherein the antibody, or antigen-binding fragment thereof, is ravulizumab administered at a dose of 500 mg, 600 mg, 700 mg, 800 mg, 900 mg, 1,000 mg, 1,100 mg, 1,200 mg, 1,300 mg, 1,400 mg, 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, or 5,000 mg.

19. The method of any one of the preceding claims, wherein:
(a) the antibody, or antigen-binding fragment thereof, is ravulizumab and the hyaluronidase is rHuPH20, and
(b) wherein the patient is administered a single formulation comprising ravulizumab at 500 mg and 10,000 units of rHuPH20.

20. The method of any one of claims 1-18, wherein:
(a) the antibody, or antigen-binding fragment thereof, is ravulizumab and the hyaluronidase is rHuPH20, and
(b) wherein the patient is administered a single formulation comprising ravulizumab at 1000 mg and 20,000 units of rHuPH20.

21. The method of any one of claims 1-18, wherein:
(a) the antibody, or antigen-binding fragment thereof, is ravulizumab and the hyaluronidase is rHuPH20, and
(b) wherein the patient is administered a single formulation comprising ravulizumab at 2000 mg and 40,000 units of rHuPH20.

22. The method of any one of the preceding claims, wherein the hyaluronidase and anti-C5 antibody, or antigen binding fragment thereof, are administered to the patient once every two weeks, once every three weeks, once a month, once every month and a half, once every two months, or once every three months.

23. The method of any one of the preceding claims, wherein the patient has been vaccinated with one or more Neisseria meningococcal vaccines prior to treatment.

24. The method of any one of the preceding claims, wherein the patient is administered an antibiotic prior to and/or during treatment.

25. The method of any one of the preceding claims, wherein the treatment results in terminal complement inhibition.

26. The method of any one of the preceding claims, wherein the treatment results in a reduction of hemolysis as assessed by lactate dehydrogenase (LDH) levels.

27. The method of any one of the preceding claims, wherein the treatment produces at least one therapeutic effect selected from the group consisting of a reduction or cessation in fatigue, abdominal pain, dyspnea, anemia, dysphagia, chest pain, and erectile dysfunction.

28. The method of any one of the preceding claims, wherein the treatment produces a shift toward normal levels of a hemolysis-related hematologic biomarker selected from the group consisting free hemoglobin, haptoglobin, reticulocyte count, PNH red blood cell (RBC) clone and D-dimer.

29. The method of any one of the preceding claims, wherein the treatment produces a shift toward normal levels of a chronic disease associated biomarker selected from the group consisting estimated glomerular filtration rate (eGFR) and spot urine:albumin:creatinine and plasma brain natriuretic peptide (BNP).

30. The method of any one of the preceding claims, wherein the treatment produces a reduction in the need for blood transfusions and/or major adverse vascular events (MAVEs).

31. The method of any one of the preceding claims, wherein the treatment produces a change from baseline in quality of life, assessed via the Functional Assessment of Chronic Illness Therapy (FACIT)-Fatigue Scale, version 4 and the European Organisation for Research and Treatment of Cancer, Quality of Life Questionnaire-Core 30 Scale.

32. The method of any one of the preceding claims, wherein the complement-associated condition is selected from the group consisting of rheumatoid arthritis, antiphospholipid antibody syndrome, lupus nephritis, ischemia-reperfusion injury, atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome.

33. The method of any one of the preceding claims, wherein the complement-associated condition is atypical hemolytic uremic syndrome (aHUS).

34. The method of any one of claims 1-32, wherein the complement-associated condition is paroxysmal nocturnal hemoglobinuria (PNH).

35. A kit for treating a complement-associated condition in a human patient, the kit comprising:
(a) a dose of an anti-C5 antibody, or antigen binding fragment thereof;
(b) a dose of a recombinant human hyaluronidase;
(c) instructions for using the anti-C5 antibody, or antigen binding fragment thereof, and recombinant human hyaluronidase in the method of any one of the preceding claims.

36. The kit of claim 35, wherein the anti-C5 antibody is ravulizumab.

37. The kit of claim 35 or 36, wherein the recombinant human hyaluronidase is rHuPH20.

38. The method of any one of claims 1-34 or the kit of any one of claims 35-37, wherein the complement-associated condition is selected from the group consisting of rheumatoid arthritis, antiphospholipid antibody syndrome, lupus nephritis, ischemia-reperfusion injury, atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome.


SEQUENCE LISTING (61 sequences)

SEQ ID NO: 1 - protein, 10 aa, Artificial Sequence
  Description of Artificial Sequence Synthetic peptide
GYIFSNYWIQ

SEQ ID NO: 2 - protein, 17 aa, Artificial Sequence
  Description of Artificial Sequence Synthetic peptide
EILPGSGSTEYTENFKD

SEQ ID NO: 3 - protein, 13 aa, Artificial Sequence
  Description of Artificial Sequence Synthetic peptide
YFFGSSPNWYFDV

SEQ ID NO: 4 - protein, 11 aa, Artificial Sequence
  Description of Artificial Sequence Synthetic peptide
GASENIYGALN

SEQ ID NO: 5 - protein, 7 aa, Artificial Sequence
  Description of Artificial Sequence Synthetic peptide
GATNLAD

SEQ ID NO: 6 - protein, 9 aa, Artificial Sequence
  Description of Artificial Sequence Synthetic peptide
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  Description of Artificial Sequence Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  Description of Artificial Sequence Synthetic peptide
GASENIYHALN

SEQ ID NO: 18 - protein, 17 aa, Artificial Sequence
  Description of Artificial Sequence Synthetic peptide
EILPGSGHTEYTENFKD

SEQ ID NO: 19 - protein, 10 aa, Artificial Sequence
  Description of Artificial Sequence Synthetic peptide
GHIFSNYWIQ

SEQ ID NO: 20 - protein, 448 aa, Artificial Sequence
  Description of Artificial Sequence Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  Description of Artificial Sequence Synthetic peptide
SYAIS

SEQ ID NO: 22 - protein, 17 aa, Artificial Sequence
  Description of Artificial Sequence Synthetic peptide
GIGPFFGTANYAQKFQG

SEQ ID NO: 23 - protein, 7 aa, Artificial Sequence
  Description of Artificial Sequence Synthetic peptide
DTPYFDY

SEQ ID NO: 24 - protein, 11 aa, Artificial Sequence
  Description of Artificial Sequence Synthetic peptide
SGDSIPNYYVY

SEQ ID NO: 25 - protein, 7 aa, Artificial Sequence
  Description of Artificial Sequence Synthetic peptide
DDSNRPS

SEQ ID NO: 26 - protein, 11 aa, Artificial Sequence
  Description of Artificial Sequence Synthetic peptide
QSFDSSLNAEV

SEQ ID NO: 27 - protein, 116 aa, Artificial Sequence
  Description of Artificial Sequence Synthetic polypeptide
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  Description of Artificial Sequence Synthetic polypeptide
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  Description of Artificial Sequence Synthetic peptide
NYIS

SEQ ID NO: 30 - protein, 17 aa, Artificial Sequence
  Description of Artificial Sequence Synthetic peptide
IIDPDDSYTEYSPSFQG

SEQ ID NO: 31 - protein, 8 aa, Artificial Sequence
  Description of Artificial Sequence Synthetic peptide
YEYGGFDI

SEQ ID NO: 32 - protein, 11 aa, Artificial Sequence
  Description of Artificial Sequence Synthetic peptide
SGDNIGNSYVH

SEQ ID NO: 33 - protein, 7 aa, Artificial Sequence
  Description of Artificial Sequence Synthetic peptide
KDNDRPS

SEQ ID NO: 34 - protein, 9 aa, Artificial Sequence
  Description of Artificial Sequence Synthetic peptide
GTYDIESYV

SEQ ID NO: 35 - protein, 116 aa, Artificial Sequence
  Description of Artificial Sequence Synthetic polypeptide
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  Description of Artificial Sequence Synthetic polypeptide
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  Description of Artificial Sequence Synthetic peptide
SSYYVA

SEQ ID NO: 38 - protein, 17 aa, Artificial Sequence
  Description of Artificial Sequence Synthetic peptide
AIYTGSGATYKASWAKG

SEQ ID NO: 39 - protein, 13 aa, Artificial Sequence
  Description of Artificial Sequence Synthetic peptide
DGGYDYPTHAMHY

SEQ ID NO: 40 - protein, 11 aa, Artificial Sequence
  Description of Artificial Sequence Synthetic peptide
QASQNIGSSLA

SEQ ID NO: 41 - protein, 7 aa, Artificial Sequence
  Description of Artificial Sequence Synthetic peptide
GASKTHS

SEQ ID NO: 42 - protein, 12 aa, Artificial Sequence
  Description of Artificial Sequence Synthetic peptide
QSTKVGSSYGNH

SEQ ID NO: 43 - protein, 123 aa, Artificial Sequence
  Description of Artificial Sequence Synthetic polypeptide
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  Description of Artificial Sequence Synthetic polypeptide
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  Description of Artificial Sequence Synthetic polypeptide
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  Description of Artificial Sequence Synthetic polypeptide
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  Description of Artificial Sequence Synthetic polypeptide
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  Description of Artificial Sequence Synthetic polypeptide
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  Description of Artificial Sequence Synthetic polypeptide
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  Description of Artificial Sequence Synthetic polypeptide
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 52 - protein, 448 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 53 - protein, 447 aa, Artificial Sequence [sequence omitted]

SEQ ID NO: 54 - protein, 446 aa, Artificial Sequence
  Description of Artificial Sequence Synthetic polypeptide
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 55 - protein, 445 aa, Artificial Sequence
  Description of Artificial Sequence Synthetic polypeptide
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 56 - protein, 444 aa, Artificial Sequence
  Description of Artificial Sequence Synthetic polypeptide
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 57 - protein, 443 aa, Artificial Sequence
  Description of Artificial Sequence Synthetic polypeptide
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 58 - protein, 442 aa, Artificial Sequence
  Description of Artificial Sequence Synthetic polypeptide
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEE

SEQ ID NO: 59 - protein, 432 aa, Artificial Sequence
  Description of Artificial Sequence Synthetic polypeptide
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 60 - protein, 448 aa, Artificial Sequence; identical to SEQ ID NO: 52
  Description of Artificial Sequence Synthetic polypeptide

SEQ ID NO: 61 - DNA, 1446 bp, Artificial Sequence
  Description of Artificial Sequence Synthetic polynucleotide
atgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctac